SEQUENCE LISTING (6 sequences)

SEQ ID NO: 1 - DNA, 5092 bp, Homo sapiens
cttggctgttcctgaggcctggcctggctccccgctgaccccttcccagacctgggatggcggaggccggcctgaggggctggctgctgtgggccctgctcctgcgcttggcccagagtgagccttacacaaccatccaccagcctggctactgcgccttctatgacgaatgtgggaagaacccagagctgtctggaagcctcatgacactctccaacgtgtcctgcctgtccaacacgccggcccgcaagatcacaggtgatcacctgatcctattacagaagatctgcccccgcctctacaccggccccaacacccaagcctgctgctccgccaagcagctggtatcactggaagcgagtctgtcgatcaccaaggccctcctcacccgctgcccagcctgctctgacaattttgtgaacctgcactgccacaacacgtgcagccccaatcagagcctcttcatcaatgtgacccgcgtggcccagctaggggctggacaactcccagctgtggtggcctatgaggccttctaccagcatagctttgccgagcagagctatgactcctgcagccgtgtgcgcgtccctgcagctgccacgctggctgtgggcaccatgtgtggcgtgtatggctctgccctttgcaatgcccagcgctggctcaacttccagggagacacaggcaatggtctggccccactggacatcaccttccacctcttggagcctggccaggccgtggggagtgggattcagcctctgaatgagggggttgcacgttgcaatgagtcccaaggtgacgacgtggcgacctgctcctgccaagactgtgctgcatcctgtcctgccatagcccgcccccaggccctcgactccaccttctacctgggccagatgccgggcagtctggtcctcatcatcatcctctgctctgtcttcgctgtggtcaccatcctgcttgtgggattccgtgtggcccccgccagggacaaaagcaagatggtggaccccaagaagggcaccagcctctctgacaagctcagcttctccacccacaccctccttggccagttcttccagggctggggcacgtgggtggcttcgtggcctctgaccatcttggtgctatctgtcatcccggtggtggccttggcagcgggcctggtctttacagaactcactacggaccccgtggagctgtggtcggcccccaacagccaagcccggagtgagaaagctttccatgaccagcatttcggccccttcttccgaaccaaccaggtgatcctgacggctcctaaccggtccagctacaggtatgactctctgctgctggggcccaagaacttcagcggaatcctggacctggacttgctgctggagctgctagagctgcaggagaggctgcggcacctccaggtatggtcgcccgaagcacagcgcaacatctccctgcaggacatctgctacgcccccctcaatccggacaataccagtctctacgactgctgcatcaacagcctcctgcagtatttccagaacaaccgcacgctcctgctgctcacagccaaccagacactgatggggcagacctcccaagtcgactggaaggaccattttctgtactgtgccaatgccccgctcaccttcaaggatggcacagccctggccctgagctgcatggctgactacggggcccctgtcttccccttccttgccattggggggtacaaaggaaaggactattctgaggcagaggccctgatcatgacgttctccctcaacaattaccctgccggggacccccgtctggcccaggccaagctgtgggaggaggccttcttagaggaaatgcgagccttccagcgtcggatggctggcatgttccaggtcacgttcatggctgagcgctctctggaagacgagatcaatcgcaccacagctgaagacctgcccatctttgccaccagctacattgtcatattcctgtacatctctctggccctgggcagctattccagctggagccgagtgatggtggactccaaggccacgctgggcctcggcggggtggccgtggtcctgggagcagtcatggctgccatgggcttcttctcctacttgggtatccgctcctccctggtcatcctgcaagtggttcctttcctggtgctgtccgtgggggctgataacatcttcatctttgttctcgagtaccagaggctgccccggaggcctggggagccacgagaggtccacattgggcgagccctaggcagggtggctcccagcatgctgttgtgcagcctctctgaggccatctgcttcttcctaggggccctgacccccatgccagctgtgcggacctttgccctgacctctggccttgcagtgatccttgacttcctcctgcagatgtcagcctttgtggccctgctctccctggacagcaagaggcaggaggcctcccggttggacgtctgctgctgtgtcaagccccaggagctgcccccgcctggccagggagaggggctcctgcttggcttcttccaaaaggcttatgcccccttcctgctgcactggatcactcgaggtgttgtgctgctgctgtttctcgccctgttcggagtgagcctctactccatgtgccacatcagcgtgggactggaccaggagctggccctgcccaaggactcgtacctgcttgactatttcctctttctgaaccgctacttcgaggtgggggccccggtgtactttgttaccaccttgggctacaacttctccagcgaggctgggatgaatgccatctgctccagtgcaggctgcaacaacttctccttcacccagaagatccagtatgccacagagttccctgagcagtcttacctggccatccctgcctcctcctgggtggatgacttcattgactggctgaccccgtcctcctgctgccgcctttatatatctggccccaataaggacaagttctgcccctcgaccgtcaactctctgaactgcctaaagaactgcatgagcatcacgatgggctctgtgaggccctcggtggagcagttccataagtatcttccctggttcctgaacgaccggcccaacatcaaatgtcccaaaggcggcctggcagcatacagcacctctgtgaacttgacttcagatggccaggttttagacacagttgccattctgtcacccaggctggagtacagtggcacaatctcggctcactgcaacctctacctcctggattcagcctccaggttcatggcctatcacaagcccctgaaaaactcacaggattacacagaagctctgcgggcagctcgagagctggcagccaacatcactgctgacctgcggaaagtgcctggaacagacccggcttttgaggtcttcccctacacgatcaccaatgtgttttatgagcagtacctgaccatcctccctgaggggctcttcatgctcagcctctgccttgtgcccaccttcgctgtctcctgcctcctgctgggcctggacctgcgctccggcctcctcaacctgctctccattgtcatgatcctcgtggacactgtcggcttcatggccctgtggggcatcagttacaatgctgtgtccctcatcaacctggtctcggcggtgggcatgtctgtggagtttgtgtcccacattacccgctcctttgccatcagcaccaagcccacctggctggagagggccaaagaggccaccatctctatgggaagtgcggtgtttgcaggtgtggccatgaccaacctgcctggcatccttgtcctgggcctcgccaaggcccagctcattcagatcttcttcttccgcctcaacctcctgatcactctgctgggcctgctgcatggcttggtcttcctgcccgtcatcctcagctacgtggggcctgacgttaacccggctctggcactggagcagaagcgggctgaggaggcggtggcagcagtcatggtggcctcttgcccaaatcacccctcccgagtctccacagctgacaacatctatgtcaaccacagctttgaaggttctatcaaaggtgctggtgccatcagcaacttcttgcccaacaatgggcggcagttctgatacagccagaggccctgtctaggctctatggccctgaaccaaagggttatggggatcttccttgtgactgccccttgacacacgccctcctcaaatcctaggggaggccattcccatgagactgcctgtcactggaggatggcctgctcttgaggtatccaggcagcaccactgatggctcctctgctcccatagtgggtccccagtttccaagtcacctaggccttgggcagtgcctcctcctgggcctgggtctggaagttggcaggaacagacacactccatgtttgtcccacactcactcactttcctaggagcccacttctcatccaacttttcccttctcagttcctctctcgaaagtcttaattctgtgtcagtaagtctttaacacgtagcagtgtccctgagaacacagacaatgaccactaccctgggtgtgatatcacaggaggccagagagaggcaaaggctcaggccaagagccaacgctgtgggaggccggtcggcagccactccctccagggcgcacctgcaggtctgccatccacggccttttctggcaagagaagggcccaggaaggatgctctcataaggcccaggaaggatgctctcataagcaccttggtcatggattagcccctcctggaaaatggtgttgggtttggtctccagctccaatacttattaaggctgttgctgccagtcaaggccacccaggagtctgaaggctgggagctcttggggctgggctggtcctcccatcttcacctcgggcctggatcccaggcctcaaaccagcccaacccgagcttttggacagctctccagaagcatgaactgcagtggagatgaagatcctggctctgtgctgtgcacataggtgtttaataaacatttgttggcagaaaaaaaaaaaaaaaaaaaaaaaaaaaaaaaaaaaaaaaaaaaaaaa

SEQ ID NO: 2 - protein, 1358 aa, Homo sapiens
MAEAGLRGWLLWALLLRLAQSEPYTTIHQPGYCAFYDECGKNPELSGSLMTLSNVSCLSNTPARKITGDHLILLQKICPRLYTGPNTQACCSAKQLVSLEASLSITKALLTRCPACSDNFVNLHCHNTCSPNQSLFINVTRVAQLGAGQLPAVVAYEAFYQHSFAEQSYDSCSRVRVPAAATLAVGTMCGVYGSALCNAQRWLNFQGDTGNGAPLDITFHLLEPGQAVGSGIQPLNEGVARCNESQGDDVATCSCQDCAASCPAIARPQALDSTFYLGQMPGSLVLIIILCSVFAVVTILLVGFRVAPARDKSKMVDPKKGTSLSDKLSFSTHTLLGQFFQGWGTWVASWPLTILVLSVIPVVALAAGLVFTELTTDPVELWSAPNSQARSEKAFHDQHFGPFFRTNQVILTAPNRSSYRYDSLLLGPKNFSGILDLDLLLELLELQERLRHLQVWSPEAQRNISLQDICYAPLNPDNTSLYDCCINSLLQYFQNNRTLLLLTANQTLMGQTSQVDWKDHFLYCANAPLTFKDGTALALSCMADYGAPVFPFLAIGGYKGKDYSEAEALIMTFSLNNYPAGDPRLAQAKLWEEAFLEEMRAFQRRMAGMFQVTFMAERSLEDEINRTTAEDLPIFATSYIVIFLYISLALGSYSSWSRVMVDSKATLGLGGVAVVLGAVMAAMGFFSYLGIRSSLVILQVVPFLVLSVGADNIFIFVLEYQRLPRRPGEPREVHIGRALGRVAPSMLLCSLSEAICFFLGALTPMPAVRTFALTSGLAVILDFLLQMSAFVALLSLDSKRQEASRLDVCCCVKPQELPPPGQGEGLLLGFFQKAYAPFLLHWITRGVVLLLFLALFGVSLYSMCHISVGLDQELALPKDSYLLDYFLFLNRYFEVGAPVYFVTTLGYNFSSEAGMNAICSSAGCNNFSFTQKIQYATEFPEQSYLAIPASSWVDDFIDWLTPSSCCRLYISGPNKDKFCPSTVNSLNCLKNCMSITMGSVRPSVEQFHKYLPWFLNDRPNIKCPKGGLAAYSTSVNLTSDGQVLDTVAILSPRLEYSGTISAHCNLYLLDSASRFMAYHKPLKNSQDYTEALRAARELAANITADLRKVPGTDPAFEVFPYTITNVFYEQYLTILPEGLFMLSLCLVPTFAVSCLLLGLDLRSGLLNLLSIVMILVDTVGFMALWGISYNAVSLINLVSAVGMSVEFVSHITRSFAISTKPTWLERAKEATISMGSAVFAGVAMTNLPGILVLGLAKAQLIQIFFFRLNLLITLLGLLHGLVFLPVILSYVGPDVNPALALEQKRAEEAVAAVMVASCPNHPSRVSTADNIYVNHSFEGSIKGAGAISNFLPNNGRQF

SEQ ID NO: 3 - DNA, 4002 bp, Mus musculus
atggcagctgcctggcagggatggctgctctgggccctgctcctgaattcggcccagggtgagctctacacacccactcacaaagctggcttctgcaccttttatgaagagtgtgggaagaacccagagctttctggaggcctcacatcactatccaatatctcctgcttgtctaataccccagcccgccatgtcacaggtgaccacctggctcttctccagcgcgtctgtccccgcctatacaatggccccaatgacacctatgcctgttgctctaccaagcagctggtgtcattagacagtagcctgtctatcaccaaggccctccttacacgctgcccggcatgctctgaaaattttgtgagcatacactgtcataatacctgcagccctgaccagagcctcttcatcaatgttactcgcgtggttcagcgggaccctggacagcttcctgctgtggtggcctatgaggccttttatcaacgcagttttgcagagaaggcctatgagtcctgtagccgggtgcgcatccctgcagctgcctcgctggctgtgggcagcatgtgtggagtgtatggctctgccctctgcaatgctcagcgctggctcaacttccaaggagacacagggaatggcctggctccgctggacatcaccttccacctcttggagcctggccaggccctggcagatgggatgaagccactggatgggaagatcacaccctgcaatgagtcccagggtgaagactcggcagcctgttcctgccaggactgtgcagcatcctgccctgtcatccctccgcccccggccctgcgcccttctttctacatgggtcgaatgccaggctggctggctctcatcatcatcttcactgctgtctttgtattgctctctgttgtccttgtgtatctccgagtggcttccaacaggaacaagaacaagacagcaggctcccaggaagcccccaacctccctcgtaagcgcagattctcacctcacactgtccttggccggttcttcgagagctggggaacaagggtggcctcatggccactcactgtcttggcactgtccttcatagttgtgatagccttgtcagtaggcctgacctttatagaactcaccacagaccctgtggaactgtggtcggcccctaaaagccaagcccggaaagaaaaggctttccatgacgagcattttggccccttcttccgaaccaaccagatttttgtgacagctaagaacaggtccagctacaagtacgactccctgctgctagggcccaagaacttcagtgggatcctatccctggacttgctgcaggagctgttggagctacaggagagacttcgacacctgcaagtgtggtcccatgaggcacagcgcaacatctccctccaggacatctgctatgctcccctcaacccgcataacaccagcctcactgactgctgtgtcaacagcctccttcaatacttccagaacaaccacacactcctgctgctcacagccaatcagactctgaatggccagacctccctggtggactggaaggaccatttcctctactgtgccaatgcccctctcacgtacaaagatggcacagccctggccctgagctgcatagctgactacggggcacctgtcttccccttccttgctgttgggggctaccaagggacggactactcggaggcagaagccctgatcataaccttctctatcaataactaccccgctgatgatccccgcatggcccacgccaagctctgggaggaggctttcttgaaggaaatgcaatccttccagagaagcacagctgacaagttccagattgcgttctcagctgagcgttctctggaggacgagatcaatcgcactaccatccaggacctgcctgtctttgccatcagctaccttatcgtcttcctgtacatctccctggccctgggcagctactccagatggagccgagttgcggtggattccaaggctactctgggcctaggtggggtggctgttgtgctgggagcagtcgtcgctgccatgggcttctactcctacctgggtgtcccctcctctctggtcatcattcaagtggtacctttcctggtgctggctgtgggagctgacaacatcttcatctttgttcttgagtaccagaggctgcctaggatgcccggggagcagcgagaggctcacattggccgcaccctgggtagtgtggcccccagcatgctgctgtgcagcctctctgaggccatctgcttctttctaggggccctgacctccatgccagctgtgaggacctttgccttgacctctggcttagcaatcatctttgacttcctgctccagatgacagcctttgtggccctgctctccctggatagcaagaggcaggaggcctctcgccccgacgtcgtgtgctgcttttcaagccgaaatctgcccccaccgaaacaaaaagaaggcctcttactttgcttcttccgcaagatatacactcccttcctgctgcacagattcatccgccctgttgtgctgctgctctttctggtcctgtttggagcaaacctctacttaatgtgcaacatcagcgtggggctggaccaggatctggctctgcccaaggattcctacctgatagactacttcctctttctgaaccggtacttggaagtggggcctccagtgtactttgacaccacctcaggctacaacttttccaccgaggcaggcatgaacgccatttgctctagtgcaggctgtgagagcttctccctaacccagaaaatccagtatgccagtgaattccctaatcagtcttatgtggctattgctgcatcctcctgggtagatgacttcatcgactggctgaccccatcctcctcctgctgccgcatttatacccgtggcccccataaagatgagttctgtccctcaacggatacttccttcaactgtctcaaaaactgcatgaaccgcactctgggtcccgtgagacccacaacagaacagtttcataagtacctgccctggttcctgaatgatacgcccaacatcagatgtcctaaagggggcctagcagcgtatagaacctctgtgaatttgagctcagatggccagattatagcctcccagttcatggcctaccacaagcccttacggaactcacaggactttacagaagctctccgggcatcccggttgctagcagccaacatcacagctgaactacggaaggtgcctgggacagatcccaactttgaggtcttcccttacacgatctccaatgtgttctaccagcaatacctgacggttctccctgagggaatcttcactcttgctctctgcttcgtgcccacctttgtggtctgctacctcctactgggcctggacatacgctcaggcatcctcaacctgctctccatcattatgatcctcgtggacaccatcggcctcatggctgtgtggggtatcagctacaatgctgtgtccctcatcaaccttgtcacggcagtgggcatgtctgtggagttcgtgtcccacattacccggtcctttgctgtaagcaccaagcctacccggctggagagagccaaagatgctactatcttcatgggcagtgcggtgtttgctggagtggccatgaccaacttcccgggcatcctcatcctgggctttgctcaggcccagcttatccagattttcttcttccgcctcaacctcctgatcaccttgctgggtctgctacacggcctggtcttcctgcccgttgtcctcagctatctggggccagatgttaaccaagctctggtactggaggagaaactagccactgaggcagccatggtctcagagccttcttgcccacagtaccccttcccggctgatgcaaacaccagtgactatgttaactacggctttaatccagaatttatccctgaaattaatgctgctagcagctctctgcccaaaagtgaccaaaagttctaa

SEQ ID NO: 4 - protein, 1332 aa, Mus musculus
MAAAWQGWLLWALLLNSAQGELYTPTHKAGFCTFYEECGKNPELSGGLTSLSNISCLSNTPARHVTGDHLALLQRVCPRLYNGPNDTYACCSTKQLVSLDSSLSITKALLTRCPACSENFVSIHCHNTCSPDQSLFINVTRVVQRDPGQLPAVVAYEAFYQRSFAEKAYESCSRVRIPAAASLAVGSMCGVYGSALCNAQRWLNFQGDTGNGLAPLDITFHLLEPGQALADGMKPLDGKITPCNESQGEDSAACSCQDCAASCPVIPPPPALRPSFYMGRMPGWLALIIIFTAVFVLLSVVLVYLRVASNRNKNKTAGSQEAPNLPRKRRFSPHTVLGRFFESWGTRVASWPLTVLALSFIVVIALSVGLTFIELTTDPVELWSAPKSQARKEKAFHDEHFGPFFRTNQIFVTAKNRSSYKYDSLLLGPKNFSGILSLDLLQELLELQERLRHLQVWSHEAQRNISLQDICYAPLNPHNTSLTDCCVNSLLQYFQNNHTLLLLTANQTLNGQTSLVDWKDHFLYCANAPLTYKDGTALALSCIADYGAPVFPFLAVGGYQGTDYSEAEALIITFSINNYPADDPRMAHAKLWEEAFLKEMQSFQRSTADKFQIAFSAERSLEDEINRTTIQDLPVFAISYLIVFLYISLALGSYSRWSRVAVDSKATLGLGGVAVVLGAVVAAMGFYSYLGVPSSLVIIQVVPFLVLAVGADNIFIFVLEYQRLPRMPGEQREAHIGRTLGSVAPSMLLCSLSEAICFFLGALTSMPAVRTFALTSGLAIIFDFLLQMTAFVALLSLDKRQEASRPDVVCCFSSRNLPPPKQKEGLLLCFFRKIYTPFLLHRFIRPVVLLLFLVLFGANLYLMCNISVGLDQDLALPKDSYLIDYFLFLNRYLEVGPPVYFDTTSGYNFSTEAGMNAICSSAGCESFSLTQKIQYASEFPNQSYVAIAASSWVDDFIDWLTPSSSCCRIYTRGPHKDEFCPSTDTSFNCLKNCMNRTLGPVRPTTEQFHKYLPWFLNDTPNIRCPKGGLAAYRTSVNLSSDGQIIASQFMAYHKPLRNSQDFTEALRASRLLAANITAELRKVPGTDPNFEVFPYTISNVFYQQYLTVLPEGIFTLALCFVPTFVVCYLLLGLDIRSGILNLLSIIMILVDTIGLMAVWGISYNAVSLINLVTAVGMSVEFVSHITRSFAVSTKPTRLERAKDATIFMGSAVFAGVAMTNFPGILILGFAQAQLIQIFFFRLNLLITLLGLLHGLVFLPVVLSYLGPDVNQALVLEEKLATEAAMVSEPSCPQYPFPADANTSDYVNYGFNPEFIPEINAASSSLPKSDQKF

SEQ ID NO: 5 - DNA, 4424 bp, Rattus norvegicus
atggcagctgcctggctgggatggctgctctgggccctgctcctgagcgcggcccagggtgagctatacacacccaaacacgaagctggggtctgcaccttttacgaagagtgcgggaaaaacccagagctctctggaggcctcacgtcactatccaatgtatcctgcctgtctaacaccccggcccgccacgtcacgggtgaacacctggctcttctccagcgcatctgtccccgcctgtacaacggccccaataccacttttgcctgttgctctaccaagcagctgctgtccttagaaagcagcatgtccatcaccaaggcccttctcacgcgctgcccggcctgctctgacaattttgtgagcttacactgccacaacacttgcagccctgaccagagcctcttcatcaacgtcacccgggtggttgagcggggcgctggagagcctcctgccgtggtggcctatgaggccttttatcagcgcagctttgctgagaaggcctatgagtcctgcagccaggtgcgcatccctgcggccgcttccttggccgtgggcagcatgtgtggagtgtatggctccgccctctgcaatgctcagcgctggctcaacttccaaggagacacagggaatggcctggctccgctggatatcaccttccacctcttggagcctggccaggccctaccggatgggatccagccactgaatgggaagatcgcaccctgcaacgagtctcagggtgatgactcagcagtctgctcctgccaggactgtgcggcgtcctgccctgtcatccctccgcccgaggccttgcgcccttccttctacatgggtcgcatgccaggctggctggccctcatcatcatcttcactgctgtctttgtgttgctctctgcagaagcccccaaactccctcataagcacaaactctcaccccataccatcctgggccggttcttccagaactggggcacaagggtggcctcgtggccactcaccgtcttagcactgtccttcatcgttgtgatagccttagcagcaggcctgacctttattgaactcaccacagaccctgtggaactgtggtcggcccccaagagccaggcccggaaagagaagtctttccatgatgagcatttcggccccttctttcgaaccaaccagattttcgtgacagctcggaacaggtccagctacaagtacgactccctactgctagggtccaagaacttcagtgggatcctgtccctggacttcctgctggagctgctggagcttcaggagaggcttcgacacctgcaagtgtggtcccctgaggcagagcgcaacatctccctccaggacatctgctatgcccccctcaacccatataacaccagcctctccgactgctgtgtcaacagcctccttcagtacttccagaacaaccgcaccctcctgatgctcacggccaaccagactctgaatggccagacctccctggtggactggaaggaccatttcctctactgtgcaaatgcccctctcacgttcaaagatggcacgtctctggccctgagctgcatggctgactacggggctcctgtcttccccttccttgctgttgggggataccaaggcacggactattccgaggcagaagcgctgatcataaccttctctctcaataactaccccgctgatgatccccgcatggcccaggccaagctctgggaggaggctttcttgaaggaaatggaatccttccagaggaacacaagtgacaagttccaggttgcgttctcagctgagcgctctctggaggatgagatcaaccgcaccaccatccaggacctgcctgtctttgccgtcagctacattatcgtcttcctgtacatctccctggccctgggcagctactccagatgcagccgagtagcggtggagtccaaggctactctgggcctaggtggggtgattgttgtgctgggagcagttctggctgccatgggcttctactcctacctgggtgtcccctcttctctggttatcatccaagtggtacctttcctggtgctagctgtgggagctgacaacatcttcatctttgttcttgagtaccagaggctacctaggatgcctggggaacagcgagaggctcacattggccgcaccctgggcagtgtggcccccagcatgctgctgtgcagcctctctgaggccatctgcttctttctaggggccctgacccccatgccagctgtgaggaccttcgccttgacctctggcttagcaattatcctcgacttcctgctccagatgactgcctttgtggccctgctctccctggatagcaagaggcaggaggcctctcgcccggatgtcttatgctgcttttcaacccggaagctgcccccacctaaagaaaaagaaggcctcttactccgcttcttccgcaagatatacgctcctttcctgctgcacagattcatccgccctgttgtgatgctgctgtttctgaccctgtttggagcaaatctctacttaatgtgcaacatcaacgtggggctagaccaggagctggctctgcccaaggactcgtacttgatagactacttcctctttctgaaccgataccttgaagtggggcctccagtgtactttgtcaccacctcgggcttcaacttctccagcgaggcaggcatgaacgccacttgctctagcgcaggctgtaagagcttctccctaacccagaaaatccagtatgccagtgaattccctgaccagtcttacgtggctattgctgcatcctcctgggtagatgacttcatcgactggctgaccccgtcctcctcctgctgtcgcctttatatacgtggcccccataaggatgagttctgtccctcaacggatacttccttcaactgcttaaaaaactgcatgaaccgcactctgggtcctgtgaggcccacagcggaacagtttcataagtacctgccctggttcctgaatgatccgcccaatatcagatgtcccaaagggggtctagcagcgtatagaacgtctgtgaatttgagctcagatggccaggttatagcctcccagttcatggcctaccacaagcccttaaggaactcacaggacttcacagaagctctccgggcgtcccggttgctagcagccaacatcacagctgacctacggaaggtgcctgggacagatccaaactttgaggtcttcccttacacgatctccaacgtgttctaccagcaatacctgacggtccttcctgagggaatcttcacccttgctctttgctttgtgcccacctttgttgtctgctacctcctactgggcctggacatgtgctcagggatcctcaacctactctccatcattatgattctcgtggacaccattggcctcatggctgtgtggggtatcagctataatgcggtatccctcatcaaccttgtcacggcagtgggcatgtctgtggagtttgtgtcccacatcactcggtcctttgctgtaagcaccaagcctacccggctggagagggctaaagatgctactgtcttcatgggcagtgcggtgtttgctggagtggccatgaccaacttcccaggcatcctcatcttgggctttgcccaagcccagcttattcagatcttcttcttccgcctcaaccttctgatcaccttgctgggtctgctgcatggcctggtcttcctgccggttgtcctcagctatctgggaccagatgttaaccaagctctggtacaggaggagaaactagccagcgaggcagcagtggccccagagccttcttgcccacagtacccctcccctgctgatgcggatgccaatgttaactacggctttgccccagaacttgcccacggagctaatgctgctagaagctctttgcccaaaagtgaccaaaagttctaatggagtaggagcttgtccatgcttcttgctgatgagggatcatgaaggtcttccctctggttgtcctcaaggcctggggggaggttgtttcagagaaaaatggctggcattcctgccacgaggcaaccggcagcattggcactgacctccttgctctcataggtccctaaggccttggtcagattacctcctccatggagagactatcttaagtatcttaagtatcgtatgggatgcatcgcctgtcaattaaaaaggctatggcctatggctcaggcagggccatccggaagaagagaggattctgggataaagccaggtgggagattcgcctggggaaaatgtgacaatggttcctgagcatgggcaatcagccatgtggcagaatgtaaattaatataaatgggttgtcttaagttatgattctagctggggaggagcctagctgtgtagccaagatatttgtaaatataaaaaaaaaaaaaaaa

SEQ ID NO: 6 - protein, 1331 aa, Rattus norvegicus
MAAAWLGWLLWALLLSAAQGELYTPKHEAGVCTFYEECGKNPELSGGLTSLSNVSCLSNTPARHVTGEHLALLQRICPRLYNGPNTTFACCSTKQLLSLESSMSITKALLTRCPACSDNFVSLHCHNTCSPDQSLFINVTRVVERGAGEPPAVVAYEAFYQRSFAEKAYESCSQVRIPAAASLAVGSMCGVYGSALCNAQRWLNFQGDTGNGLAPLDITFHLLEPGQALPDGIQPLNGKIAPCNESQGDDSAVCSCQDCAASCPVIPPPEALRPSFYMGRMPGWLALIIIFTAVFVLLSAVLVRLRVVSNRNKNKAEGPQEAPKLPHKHKLSPHTILGRFFQNWGTRVASWPLTVLALSFIVVIALAAGLTFIELTTDPVELWSAPKSQARKEKSFHDEHFGPFFRTNQIFVTARNRSSYKYDSLLLGSKNFSGILSLDFLLELLELQERLRHLQVWSPEAERNISLQDICYAPLNPYNTSLSDCCVNSLLQYFQNNRTLLMLTANQTLNGQTSLVDWKDHFLYCANAPLTFKDGTSLALSCMADYGAPVFPFLAVGGYQGTDYSEAEALIITFSLNNYPADDPRMAQAKLWEEAFLKEMESFQRNTSDKFQVAFSAERSLEDEINRTTIQDLPVFAVSYIIVFLYISLALGSYSRCSRVAVESKATLGLGGVIVVLGAVLAAMGFYSYLGVPSSLVIIQVVPFLVLAVGADNIFIFVLEYQRLPRMPGEQREAHIGRTLGSVAPSMLLCSLSEAICFFLGALTPMPAVRTFALTSGLAIILDFLLQMTAFVALLSLDSKRQEASRPDVLCCFSTRKLPPPKEKEGLLLRFFRKIYAPFLLHRFIRPVVMLLFLTLFGANLYLMCNINVGLDQELALPKDSYLIDYFLFLNRYLEVGPPVYFVTTSGFNFSSEAGMNATCSSAGCKSFSLTQKIQYASEFPDQSYVAIAASSWVDDFIDWLTPSSSCCRLYIRGPHKDEFCPSTDTSFNCLKNCMNRTLGPVRPTAEQFHKYLPWFLNDPPNIRCPKGGLAAYRTSVNLSSDGQVIASQFMAYHKPLRNSQDFTEALRASRLLAANITADLRKVPGTDPNFEVFPYTISNVFYQQYLTVLPEGIFTLALCFVPTFVVCYLLLGLDMCSGILNLLSIIMILVDTIGLMAVWGISYNAVSLINLVTAVGMSVEFVSHITRSFAVSTKPTRLERAKDATVFMGSAVFAGVAMTNFPGILILGFAQAQLIQIFFFRLNLLITLLGLLHGLVFLPVVLSYLGPDVNQALVQEEKLASEAAVAPEPSCPQYPSPADADANVNYGFAPELAHGANAARSSLPKSDQKF

(19) United States
(12) Patent Application Publication
(10) Pub. No.: US 2010/0119525 A1
(43) Pub. Date: May 13, 2010

(54) METHOD FOR EXTENDING LONGEVITY USING NPC1L1 ANTAGONISTS

(75) Inventor: Yiannis Ioannou, New York, NY (US)

Correspondence Address: DARBY & DARBY P.C., P.O. BOX 770, Church Street Station, New York, NY, 10008-0770, US

(73) Assignee: MOUNT SINAI SCHOOOL OF MEDICINE OF NEW YORK UNIVERSITY, NEW YORK, NY (US)

(21) Appl. No.: 11/997,600

(22) PCT Filed: Aug. 1, 2006
(86) PCT No.: PCT/US06/30162

§ 371 Date: Nov. 10, 2008

Related U.S. Application Data
(60) Provisional application No. 60/704,759, filed on Aug. 1, 2005.

(51) Int. Cl. A61K 31/397; A61K 39/395; A61K 31/7088; A61K 31/7105; A61K 31/445; A61K 31/497; A61K 31/495; A61K 31/381; A61K 31/135; A61K 31/405; A61K 31/56; A61K 38/22; A61K 31/40; A61K 31/44; A61K 31/24; A61K 31/66; A61K 31/60

(52) U.S. Cl. 424/172.1; 514/210.02; 514/44 A; 514/331; 514/252.12; 514/330; 514/445; 514/646; 514/448; 514/654; 514/415; 514/171; 514/12; 514/423; 514/283; 514/537; 514/114; 514/165

(57) ABSTRACT

The present invention relates to a method for prolonging longevity using and NPC1L1 antagonist. The present invention also provides a method for reducing weight in an individual who consumes a high-fat diet using an NPC1L1 antagonist.

### CROSS-REFERENCE TO RELATED APPLICATIONS

Priority is claimed under 35 U.S.C. §119(e) to co-pending U.S. Provisional Patent Application Ser. No. 60/704,759, filed on Aug. 1, 2005. The contents of this priority application is hereby incorporated into the present disclosure by reference and in its entirety.

### STATEMENT REGARDING FEDERALLY SPONSORED RESEARCH

The research leading to this invention was supported, in at least part, by the National Institutes of Health, Grant No. DK 54736. Accordingly, the United States government may have certain rights to this invention.

### FIELD OF INVENTION

The present invention relates to a method for prolonging longevity using and NPC1L1 antagonist. The present invention also provides a method for reducing weight in an individual who consumes a high-fat diet using an NPC1L1 antagonist.

### BACKGROUND OF THE INVENTION

### Niemann Pick C1

The human Niemann-Pick C1 gene (NPC1) encodes a receptor responsible for the rare cholesterol storage disease, Niemann-Pick C1. NPC1 localizes to late endosomes and plays a role in intracellular transport of cholesterol. Cells lacking NPC1 have a number of distinct trafficking defects: (i) unesterified cholesterol derived from low-density lipoproteins (LDLs) accumulates in lysosomes; (ii) cholesterol accumulates in the trans-golgi network; and (iii) cholesterol transport to and from the plasma membrane is delayed.

Previously, a novel Niemann-Pick C1 Like 1 (NPC1L1) gene was identified that is also involved in lipid metabolism (Davies et al, Genomics. 2000; 65(2):137-45). This gene and corresponding gene product are described in commonly-owned provisional patent application Ser. No. 60/592,592, filed on Jul. 30, 2004, and WO 2006/015365 (corresponding to International Patent Application No. PCT/US2005/027579, filed Aug. 1, 2005) the disclosures of which are hereby incorporated by reference in their entireties. NPC1L1 co-localizes with the GTPase Rab 5 in HepG2 cells (Davies et al., J. Biol. Chem. 2005; 280: 12710-20). These data indicate that NPC1L1 is an internal membrane resident protein that appears to share lipid permease activity with its homolog NPC1.

It has recently been shown that NPC1L1 is critical for cholesterol absorption via the lumen of the small intestine in humans and mice (Altmann et al., Science. 2004; 303: 1201-04). Specifically, NPC1L1 is highly expressed in the brush border of enterocytes, in the epithelial layer of the jejunum proximal to the intestinal lumen. In contrast to the study referenced above, other studies have suggested that NPC1L1 is expressed in the plasma membranes of enterocyes, not intracelularly, and transports cholesterol from the intestinal lumen into tissue. NPC1L1 is also highly expressed in the liver in humans but not in mice. NPC1L1 has also been shown to be a target of the anti-cholesterol drug ezetimibe, which inhibits transport of dietary and biliary cholesterol in the intestinal lumen, (Garcia-Calvo et al., Proc. Natl. Acad. Sci. USA. 2005; 102: 8132-37). This is supported by the fact that NPC1L1−/− mice were unaffected by treatment with ezetimibe (Altmann et al., supra). Some evidence suggests that NPC1L1 functions within a multi-protein complex to transport cholesterol.

In addition, activation of nuclear receptor peroxisome proliferator-activated receptor sigma (PPARδ), known to be involved in increasing plasma high density lipoprotein (HDL), has been shown to induce an increase in fecal neutral sterol loss, i.e., excretion of lipid such as cholesterol (Jelske et al., J. Lipid Research. 2005; 46: 526-34). This observation was correlated with decreased expression of NPC1L1 in the intestine, suggesting that reduced cholesterol absorption through the intestine contributes to the increased cholesterol excretion, and thus, anti-artherosclerotic effect, of PPARδ activation. Interestingly, no increase in biliary secretion of cholesterol, despite increased HDL levels, was observed upon PPARδ activation.

Provisional application 60/592,592, filed Jul. 30, 2004, and WO 2006/015365 (corresponding to International Patent Application No. PCT/US2005/027579, filed Aug. 1, 2005) also described the generation of NPC1L1 knockout mice (NPC1L1−/−) in the C57BL6 strain. The resulting NPC1L1−/− mice were found to breed normally and showed no obvious phenotype when compared with their wild-type NPC1L1+/+ counterparts. However, the NPC1L1−/− mice exhibit a substantial reduction in absorbed cholesterol (Altmann et al., supra), and are completely resistant to diet-induced hypercholesterolemia (Davis et al., J. Biol. Chem. 2004; 279: 33586-33592). Recently, the NPC1L1−/− mice were further characterized and shown to exhibit defects in plasma membrane uptake and intracellular transport of lipids, including cholesterol and sphingolipids, and also exhibit defects in caveolin transport and localization (Davies et al., J. Biol. Chem. 2005; 280: 12710-20). These studies also support the finding that NPC1L1 is not localized to the plasma membrane, but instead resides intracellularly in internal membranes.

### Caloric Restriction and Long Life

It is well established in lower organisms that nutrient-balanced caloric restriction (CR) in healthy (i.e., non-obese) individuals has the unexpected benefit of extending life span. CR induces metabolic changes, improves insulin sensitivity and alters neuroendocrine function in animals (Bordone et al., Nat Rev Mol Cell Biol. 2005; 6(4):298-305). Rodents and primates on calorie-restricted diets exhibit lower plasma insulin levels and greater insulin sensitivity; lower body temperatures; reduced cholesterol, triglycerides, blood pressure, and arterial stiffness; elevated HDL; and slower age-related decline in circulating levels of DHEAS (Roth et al., Ann NY Acad Sci. 2001; 928:305-15). In addition, such life extension has been observed over the years in many other species, including humans, hamsters, dogs, fish, invertebrate animals, and yeast (Masoro, Mech Ageing Dev. 2005; 126: 913-22).

The underlying mechanism of the beneficial life-prolonging effects of CR is not well characterized. It has been suggested that the beneficial effects of intermittent fasting and CR result from a combination of reduced oxidative damage and increased cellular stress resistance (Mattson et al., J Nutr Biochem. 2005; 16(3):129-37). One study has linked the improvement of glutamate dysregulation, mitochondrial dysfunction and protein synthesis by CR is, least partially, to the CR-mediated alteration of the oxidation or the expression of certain genes in hippocampal and striatal brains of rats (Poon et al., Neurobiol Aging. 2006; 27(7): 1010-9 and Poon et al., Neurobiol Aging. 2006; 27(7): 1020-34). In another study, rats subjected during 1 year to 40% CR starting at 24 months of age exhibited a significantly decreased rate of mitochondrial H₂O₂ production (by 24%) and oxidative damage to mtDNA (by 23%) in the brain. These levels were below the level of both old and young ad libitum-fed animals (Sanz et al., J Bioenerg Biomembr. 2005; 37(2):83-90). Moreover, oxidative damage to nuclear DNA increased with age and this increase was fully reversed by CR to the level of the young controls.

In yeast, the SIR2 gene mediates the life-extending effects of calorie restriction. Recently, the mammalian SIR2 orthologue, SIRT1 (sirtuin 1) was shown to be induced in CR rats (Cohen et al., Science. 2004; 305(5682):390-2). Insulin and insulin-like growth factor 1 (IGF-1) attenuated this response. More recently, circulating insulin and IGF-1 levels were shown reduced by CR in normal mice, which also demonstrated increased protein levels of SIRT1 (Al-Regaiey et al., Endocrinology. 2005; 146(2):851-60). SIRT1 activation also was shown to repress peroxisome proliferator-activated receptor gamma transactivation and inhibits lipid accumulation in adipocytes (Picard et. al., Nature. 2004; 429(6993):771-6). It was hypothesized that the effect of adipose tissue reduction on lifespan could be due to the production of adipokines acting on target tissues such as the brain, or due to the indirect prevention of age-related metabolic disorders like type 2 diabetes or atherosclerosis (Picard et al., Int J Obes Relat Metab Disord. 2005; 29 Suppl 1:S36-9).

SIRT1 has also been linked to oxidative stress by regulating members of the forkhead transcription factor, group O (FOXO) transcription factor family, which in turn, regulates genes related to stress resistance (Kobayashi et al., Int J Mol Med. 2005; 16(2):237-43). The various adverse processes activated upon FOXO suppression include increased generation of reactive oxygen species (ROS; Morris et al., J Hypertens. 2005; 23(7):1285-309). One hypothesis regarding the benefit of CR is attributed to the consequence of an active cellular response to a low-intensity stress (Anderson et al., Nature. 2003 May 8; 423(6936):181-5). Supporting this, it was demonstrated that small molecule activators of yeast homolog of SIRT1 extends the lifespan of yeast by 70% (Howitz et al., Nature. 2003; 25(6954):191-6).

As further support for the insulin sensitivity modulation hypothesis, fasting of obese, insulin resistant mice resulted in increased expression Of hepatic genes that participate in pathways responsible for modulating insulin sensitivity (Raab al., Nutr Metab (Lond). 2005; 2(1):15). Similar results were obtained in studies of hypoinsulinemic dwarf mice subjected to CR via feeding on alternate days only (Mastemak et al., Exp Gerontol. 2005; 40(6):491-7). This suggests that some of the benefits of CR are due to effects on modulating insulin sensitivity. In primates, the physiological effects of CR seen in short-lived animals were improved glucose metabolism and altered insulin sensitivity, altered secretion of many hormones, and altered gene-expression profile of cells in the muscle, heart, and brain (Armandola, Highlights of the 5th European Molecular Biology Organization Interdisciplinary Conference on Science and Society—Time & Aging: Mechanisms and Meanings; Nov. 5-6, 2004; Heidelberg, Germany).

In addition, also CR benefits unhealthy individuals, i.e., obese individuals or individuals with cardiovascular disorders such as dyslipidemia, or other chronic diseases. For example, increased morbidity associated with obesity and aging is largely due to cardiovascular disease. Total and HDL cholesterol are major determinants of coronary heart disease. Saturated and trans fatty acids have a total and LDL cholesterol elevating effect, while unsaturated fatty acids have a lowering effect (Kromhout, J Nutr Health Aging. 2001; 5(3):144-9). According to the American Heart Association 2001 Heart and Stroke Statistical Update, atherosclerosis, the pathological accumulation of fatty acids such as cholesterol, on arterial walls, accounted for 75% of all deaths due to cardiovascular diseases (Subramanian et al., Indian J Exp Biol. 2003; 41(1):14-25). In addition, several studies have demonstrated that the predominance of the larger, more lipid-rich HDL2 subclass is a reproducible phenotype among people who have reached the age of 100 (reviewed in Arai et al., J Atheroscler Thromb. 2004; 11(5):246-52).

For obese individuals, benefits that result from caloric restriction (resulting from bariatric surgery) include loss of fat mass. Fat mass participates in glucose metabolism through the release of adipocytokines, which favorably impacts insulin resistance (Gumbs et al., Obes Surg. 2005; 15(4):462-73).

A recent hypothesis attributes CR to positive benefits of cellular homeostasis and a reduction in cell stress. This is supported by recent findings that suggest that rate of aging is determined not by metabolic rate but by metabolic stability, which is a measure of a cell's ability to maintain stable ratios of certain critical cellular metabolites in the face of stress. Otherwise, the cell's function is compromised (Demetrius et al., J Gerontol A Biol Sci Med Sci. 2004; 59(9):B902-15). Further, other published studies demonstrated that aging was accompanied by changes in gene expression associated with increased inflammation, cellular stress and fibrosis, and reduced capacity for apoptosis, xenobiotic metabolism, normal cell-cycling, and DNA replication (Spindler Mech Ageing Dev. 2005; 126(9): 960-6). Long-term calorie restriction and just 4 weeks of short-term calorie restriction reversed the majority of these changes and produced a gene expression profile consistent with decreased inflammation and cellular stress, increased apoptosis, improved metabolism of foreign chemicals, and better cardiovascular health.

In addition, a positive influence between stress in the endoplasmic reticulum (ER) and CR has been established. The ER is responsible for secretory protein folding (rough ER), and is the site of cellular lipid and cholesterol synthesis (smooth ER). The ER has an established stress response in response to toxic accumulation of misfolded proteins, in oxidative stress, calcium dysregulation, or lipid accumulation. CR has been shown to reduce the expression of genes regulating the ER stress response, as well as reduce the expression of the ER molecular chaperones which assist protein folding (Spindler et al., Biochem. Biophys. Res. Comm. 2001; 284: 335-39).

Further, decreased molecular chaperone expression was shown to increase serum protein secretion from the liver. Increased serum protein secretion should clear old, glycosylated proteins from the blood, which prevents tissue and vessel damage resulting from Maillard “browning” reactions of the sugars on the proteins with body tissue. Such Maillard reactions cause micro- and macrovascular damage that is associated with diseases including kidney disease, neurological diseases, visual disease, coronary disease and diabetes. Further, the ER stress response correlates the insulin and post-prandial increase in protein synthesis with levels of ER chaperones. The hypothesis is that reducing calories will also positively impact ER stress and improve protein clearance from the blood.

### SUMMARY OF THE INVENTION

The present invention provides a method for extending longevity in an individual comprising administering to the individual an effective amount of an NPC1L1 antagonist.

In one embodiment, the individual has not been diagnosed with a chronic disorder which adversely impacts longevity, such as hypercholesterolemia or dyslipidemia.

In another embodiment, the individual has been diagnosed with a chronic disorder which adversely impacts longevity, such as hypercholesterolemia or dyslipidemia.

In this embodiment, the chronic disorder is a cardiovascular disease, such as hyperlipidemia, dyslipidemia, and artherosclerosis.

In a specific embodiment of the invention, the NPC1L1 antagonist is ezetimibe (Zetia®).

In some embodiments, ezetimibe (Zetia®) is administered at a dose of about 0.5 to 20 mg/day, or about 5-15 mg/day.

In a particular embodiment, ezetimibe (Zetia®) is administered at a dose of about 10 mg/day.

In another embodiment, the NPC1L1 antagonist is a 4-phenylpiperidine.

In a specific embodiment, the NPC1L1 antagonist is 4-phenyl-4-piperidinecarbonitrile hydrochloride.

In further embodiment, the NPC1L1 antagonist is 1-butyl-N-(2,6-dimethylphenyl)-2-piperidinecarboxamide.

In still another embodiment, the NPC1L1 antagonist is 1-(1-napthylmethyl)-piperazine.

In other embodiments, the NPC1L1 antagonist is selected from the group consisting of an anti-NPC1L1 antibody, an NPC1L1 antisense nucleic acid, an NPC1L1 ribozyme, an NPC1L1 triple-helix, or an NPC1L1 inhibitory RNA.

In other embodiment the transcription of NPC1L1 mRNA is inhibited by targeting NPC1L1 promoter transcription factors using an antagonist(s) to these factors. In this embodiment the specific antagonist is identified by its ability to downregulate the expression of a reporter gene (such as luciferase or green fluorescence protein) driven by the mouse, rat or human promoter for NPC1L1.

In a specific embodiment, the NPC1L1 antagonist is an inhibitory RNA.

In another embodiment, the longevity is prolonged by at least 15% compared to the expected longevity of the individual, or as compared to an individual of similar expected longevity who has not been administered an NPC1L1 antagonist.

The present invention further provides a method of prolonging longevity by administering the NPC1L1 antagonist in combination with a second, longevity-prolonging agent.

In some embodiments, the second, longevity-prolonging agent is selected from the group consisting of deprenyl, melatonin, centrophenoxine, dehydroepiandrosterone (DHEA), synthetic human growth hormone, piracetam, vinpocetine-hydergine, procaine, centrophenoxine, phosophatidylserine, acetyl-L-carnatine, aspirin, and inhibitors of reactive oxygen intermediates.

The present invention also provides a method for reducing weight in an individual who consumes a high-fat diet, by administering an NPC1L1 antagonist in a pharmaceutically acceptable carrier.

In one embodiment, the individual exhibits reduced food consumption.

In a second embodiment, the individual does not exhibit reduced food consumption.

In one embodiment, the NPC1L1 antagonist is ezetimibe (Zetia®).

In another embodiment, the NPC1L1 antagonist is a 4-phenylpiperidine.

In a specific embodiment of the above, the NPC1L1 antagonist is 4-phenyl-4-piperidinecarbonitrile hydrochloride.

In further embodiment, the NPC1L1 antagonist is 1-butyl-N-(2,6-dimethylphenyl)-2-piperidinecarboxamide.

In still another embodiment, the NPC1L1 antagonist is 1-(1-napthylmethyl)-piperazine.

In another embodiment, the inhibitor is 4-butyrl-4-phenylpiperidine hydrochloride. In a further embodiment, the NPC1L1 antagonist is 3-{1-[2-methylphenyl)amino]ethylidene}-2,4-(3H,5H)-thiopenedione.

In a further embodiment, the NPC1L1 antagonist is 3-{1-[2-hydroxyphenyl)amino]ethylidene}-2,4-(3H,5H)-thiophenedione.

In a further embodiment, the NPC1L1 antagonist is 2-acetyl-3-[2-methylphenyl)amino]-2-cyclopenten-1-one.

In a further embodiment, the NPC1L1 antagonist is 3-[4-methoxyphenyl)amino]-2-methyl-2-cyclopenten-1-one.

In a further embodiment, the NPC1L1 antagonist is 3-[2-methoxyphenyl)amino]-2-methyl-2-cyclopenten-1-one.

In a further embodiment, the NPC1L1 antagonist is N-(4-acetylphenyl)-2-thiophenecarboxamide.

### DETAILED DESCRIPTION

The present invention relates to the unexpected discovery that the NPC1L1−/− mice live at least 15% longer than their wild-type NPC1L1+/+ counterparts. Accordingly, the present invention provides a method for extending the life of an individual by administering NPC1L1 gene or protein antagonists.

In view of the beneficial effects of CR discussed above, any therapy that provides similar benefits to CR would also benefit a population of individuals with cardiovascular disease. Since the present inventors identified NPC1L1 as a regulator of multiple lipid uptake and transport, it was proposed that NPC1L1 inactivation may have an effect on extending or prolonging longevity by having an effect similar to caloric restriction.

Although the mechanism by which NPC1L1 inhibition is not yet defined, the method of the invention does not depend on knowing the precise mechanism. The mechanism may be related to the above-discussed hypotheses and findings for CR, it may or may not be related to the cholesterol transport inhibition, or it may be due to and entirely different mechanism of action (discussed below). It is sufficient for the methods of the present invention that a link is established between NPC1L1 inhibition and extended longevity.

### DEFINITIONS

Niemann-Pick C1 like gene (NPC1L1; NPC3) refers to an Niemann Pick C1-like gene and gene product. The sequence for human NPC1L1 is obtained in Genbank Accession No. AF192522 and in SEQ ID NO: 1 (Davies et al., Genomics. 2000; 65(2): 137-145 and Ioannou et al., Mod. Genet. Metab. 2000; 71(1-2): 175-181). The Niemann Pick C1-like protein was first isolated in humans, based on its 42% amino acid identity and 51% amino acid similarity to human NPC1. The amino acid sequence of human NPC1L1 is depicted in Genbank Accession No. AF002020 (SEQ ID NO: 2). This term also refers to orthologs such as described in GenBank Accession Nos.: AY437866 (SEQ ID NO: 3 and SEQ ID NO; 4; mouse NPC1L1 nucleotide and amino acid, respectively); AY437867 (SEQ ID NO: 5 and SEQ ID NO: 6; rat NPC1L1 nucleotide and amino acid, respectively);

The terms “prolonging life” or “prolonging longevity” or “extending life” or “extending longevity” refer to extending the lifespan of an individual upon administration of an NPC1L1 antagonist as compared to the expected lifespan of an individual in similar health who is not being administered an NPC1L1 antagonist, or as compared to the individual if s/he had not been administered the NPC1L1 antagonist. Prolonged life can be determined using any of the methods described above.

In one embodiment of the invention, the individual being administered an NPC1L1 antagonist does not have a chronic disease or condition that adversely impacts life expectancy, such as, but not limited to, dyslipidemia (i.e., artherosclerosis, high cholesterol, elevated LDL cholesterol, reduced HDL cholesterol, or elevated triglycerides, or any combination of the foregoing), diabetes, obesity, or cancer. In other words, the individual is considered healthy and has a life expectancy within the estimated median (considering age, race, gender, demographic location, etc.).

In another embodiment of the invention, the individual being administered an NPC1L1 antagonist for extending or prolonging life does present with a chronic disease or condition which adversely impacts life expectancy. In this embodiment, the life expectancy of an individual being administered the NPC1L1 antagonist will be extended as compared to a similarly situated (i.e., an individual with the chronic disease and a similar estimated life expectancy) individual not being administered an NPC1L1 antagonist, or as compared to the life expectancy of the individual has s/he not been administered an NPC1L1 antagonist. This population includes individuals with dyslipidemia (as described above) or obesity.

These terms do not apply to individuals having acute, life-threatening diseases or disorders, such as infectious diseases, or to life-threatening complications resulting from chronic diseases (such as opportunistic infections resulting from AIDS). Nor does this term apply to individuals who have been traumatically injured as a result of accidents, injuries, force mejeure or the like (e.g., tsunamis) that adversely impact expected lifespan.

Ezetimibe (Zetia®) refers to 1-(4-fluorophenyl)-(3R)-[3-(4-fluorophenyl)-3(S)-hydroxypropyl]-4(S)-(4-hydroxyphenyl)-2-azetidinone. Etezimibe has the following structural formula:

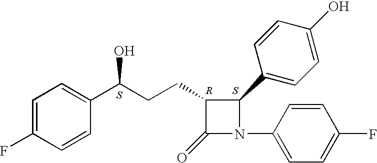

Zetia® is available in 10 mg tablets for oral administration, and is manufactured by Merck/Schering Plough (North Wales, Pa.). Zetia increases HDL cholesterol, and reduces total and low density lipoproteins (LDS) by inhibiting absorption through the small intestine. The cholesterol then bypasses the liver and instead is excreted.

A second NCP1L1 antagonist contemplated is 4-phenyl-4-piperidinecarbonitrile hydrochloride, which has the following structure:

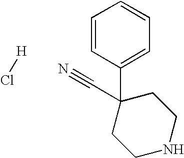

A third NPC1L1 inhibitor contemplated is 1-(1-napthylmethyl)-piperazine, having the following structure:

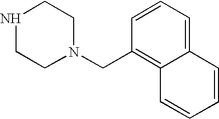

A fourth NPC1L1 inhibitor contemplated for use according to the present invention is 1-Butyl-N-(2,6-dimethylphenyl)-2-piperidinecarboxamide, which has the following structure:

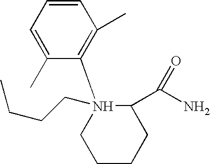

A fifth NPC1L1 antagonist contemplated for use according to the present invention is 4-butyrl-4-phenylpiperidine hydrochloride, which has the following structure:

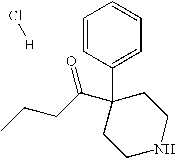

The term “subject” or “individual” as used herein refers to a mammal (e.g., a rodent such as a mouse or a rat, a pig, a primate, or companion animal (e.g., dog or cat, etc.). In particular, the term refers humans.

An NPC1L1 antagonist refers to an agent that reduces expression or activity, or inhibits expression or activity, of an NPC1L1 nucleic acid or polypeptide. Examples of antagonists of the NPC1L1-encoding nucleic acids of the present invention include without limitation antisense nucleic acids, ribozymes, RNAi oligonucleotides; small molecule drug compounds such as ezetimibe (described infra), and peptides and petide mimetics.

An “antisense” nucleic acid molecule or oligonucleotide is a single stranded nucleic acid molecule, which may be DNA, RNA, a DNA-RNA chimera, or a derivative thereof, which, upon hybridizing under physiological conditions with complementary bases in an RNA or DNA molecule of interest, inhibits the expression of the corresponding gene by inhibiting, e.g., mRNA transcription, mRNA splicing, mRNA transport, or mRNA translation or by decreasing mRNA stability. As presently used, “antisense” broadly includes RNA-RNA interactions, RNA-DNA interactions, and RNase-H mediated arrest. Antisense nucleic acid molecules can be encoded by a recombinant gene for expression in a cell (see, e.g., U.S. Pat. Nos. 5,814,500 and 5,811,234), or alternatively they can be prepared synthetically (see, e.g., U.S. Pat. No. 5,780,607). According to the present invention, the role of NPC1L1 in regulation of condition associate with hyperlipidemia may be identified, modulated and studied using antisense nucleic acids derived on the basis of NPC1L1-encoding nucleic acid molecules of the invention.

The term “ribozyme” is used to refer to a catalytic RNA molecule capable of cleaving RNA substrates. Ribozyme specificity is dependent on complementary RNA-RNA interactions (for a review, see Cech and Bass, Annu. Rev. Biochem. 1986; 55: 599-629). Two types of ribozymes, hammerhead and hairpin, have been described. Each has a structurally distinct catalytic center. The present invention contemplates the use of ribozymes designed on the basis of the NPC1L1-encoding nucleic acid molecules of the invention to induce catalytic cleavage of the corresponding mRNA, thereby inhibiting expression of the NPC1L1 gene. Ribozyme technology is described further in Intracellular Ribozyme Applications Principals and Protocols, Rossi and Couture ed., Horizon Scientific Press, 1999.

The term “RNA interference” or “RNAi” refers to the ability of double stranded RNA (dsRNA) to suppress the expression of a specific gene of interest in a homology-dependent manner. It is currently believed that RNA interference acts post-transcriptionally by targeting mRNA molecules for degradation. RNA interference commonly involves the use of dsRNAs that are greater than 500 bp; however, it can also be mediated through small interfering RNAs (siRNAs) or small hairpin RNAs (shRNAs), which can be 10 or more nucleotides in length and are typically 18 or more nucleotides in length. For reviews, see Bosner and Labouesse, Nature Cell Biol. 2000; 2: E31-E36 and Sharp and Zamore, Science 2000; 287: 2431-2433.

The term “inhibiting NPC1L1 transcription” refers to administering any agent that blocks NPC1L1 expression at the transcriptional level. Such an agent can achieve this by binding to the NPC1L1 promoter and/or enhancer and inhibiting binding of transcription factors required for NPC1L1 expression, or by binding to and inhibiting a factor, such as inhibiting a transcription factor or other agent that binds to the NPC1L1 promoter and/or enhancer, or binds to a complex of required factors, which is required for NPC1L1 expression (transcription).

A “therapeutically effective amount” or “effective dose” refer to the amount of the NPC1L1 antagonist that is sufficient to result in a therapeutic response. A therapeutic response may be any response that a user (e.g., a clinician) will recognize as an effective response to the therapy, including the foregoing symptoms and surrogate clinical markers. Thus, a therapeutic response will generally be an amelioration of one or more symptoms of a disease or disorder, or, will provide a recognizable and measurable beneficial effect. As used herein, an effective amount of an NPC1L1 antagonist is an amount that reduces NPC1L1 expression or activity to sufficiently to prolong or extend life or longevity beyond that which is expected in the absence of being administered the NPC1L1 antagonist.

The phrase “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to +5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

A “test compound” is a molecule that can be tested for its ability to act as an antagonist of an NPC1L1 gene or gene product. Test compounds can be selected without limitation from small inorganic and organic molecules (i.e., those molecules of less than about 2 kD, and more preferably less than about 1 kD in molecular weight), polypeptides (including native ligands, antibodies, antibody fragments, and other immunospecific molecules), oligonucleotides, polynucleotide molecules, and derivatives thereof. In various embodiments of the present invention, a test compound is tested for its ability to reduce or inhibit the expression and/or activity of a mammalian NPC1L1-encoding nucleic acid or NPC1L1 protein or to bind to a mammalian NPC1L1 protein. A compound that modulates a nucleic acid or protein of interest is designated herein as a “candidate compound” or “lead compound” suitable for further testing and development. Candidate compounds include, but are not necessarily limited to, the functional categories of agonist and antagonist.

As used herein, the terms “transfected cell” and “transformed cell” both refer to a host cell that has been genetically modified to express or over-express a nucleic acid encoding a specific gene product of interest such as, e.g., a NPC1L1 protein or a fragment thereof. Any eukaryotic or prokaryotic cell can be used, although eukaryotic cells are preferred, vertebrate cells are more preferred, and mammalian cells are the most preferred. In the case of multi-subunit protein complexes, nucleic acids encoding the several subunits are preferably co-expressed by the transfected or transformed cell to form a functional channel. Transfected or transformed cells are suitable to conduct an assay to screen for compounds that modulate the function of the gene product. A typical “assay method” of the present invention makes use of one or more such cells, e.g., in a microwell plate or some other culture system, to screen for such compounds. The effects of a test compound can be determined on a single cell, or on a membrane fraction prepared from one or more cells, or on a collection of intact cells sufficient to allow measurement of activity.

In accordance with the present invention, there may be employed conventional molecular biology, microbiology, and recombinant DNA techniques within the skill of the art. See, e.g., Sambrook, Fritsch and Maniatis, Molecular Cloning: A Laboratory Manual, 2^{nd} ed., Cold Spring Harbor Laboratory Press, Cold Spring Harbor, N.Y., 1989 (herein “Sambrook et al., 1989”); DNA Cloning: A Practical Approach, Volumes I and II (Glover ed. 1985); Oligonucleotide Synthesis (Gait ed. 1984); Nucleic Acid Hybridization (Hames and Higgins eds. 1985); Transcription And Translation (Hames and Higgins eds. 1984); Animal Cell Culture (Freshney ed. 1986); Immobilized Cells And Enzymes (IRL Press, 1986); B. Perbal, A Practical Guide To Molecular Cloning (1984); Ausubel et al. eds., Current Protocols in Molecular Biology, John Wiley and Sons, Inc. 1994; Sambrook et al. (2001) Molecular Cloning: A Laboratory Manual. 3^{rd} ed. Cold Spring Harbor Laboratory Press: Cold Spring Harbor, N.Y. (herein “Sambrook et al., 2001”); Ausubel et al. eds. (2006) Current Protocols in Molecular Biology. John Wiley and Sons, Inc.: Hoboken, N.J.; Bonifacino et al. eds. (2006) Current Protocols in Cell Biology. John Wiley and Sons, Inc.: Hoboken, N.J.; Coligan et al. eds. (2006) Current Protocols in Immunology, John Wiley and Sons, Inc.: Hoboken, N.J.; Coico et al. eds. (2006) Current Protocols in Microbiology, John Wiley and Sons, Inc.: Hoboken, N.J.; Coligan et al. eds. (2006) Current Protocols in Protein Science, John Wiley and Sons, Inc.: Hoboken, N.J.; Enna et al. eds. (2006) Current Protocols in Pharmacology John Wiley and Sons, Inc.: Hoboken, N.J.; Hames et al. eds. (1999) Protein Expression: A Practical Approach. Oxford University Press: Oxford; Freshney (2000) Culture of Animal Cells: A Manual of Basic Technique. 4^{th} ed. Wiley-Liss; among others (the Current Protocols listed above are updated several times every year); among others.

### NPC1L1 Inhibition and Longevity

Since NPC1L1 appears to regulate the flow of lipids (and possibly other nutrients) from the plasma membrane (uptake) to the various cellular organelles such as Golgi and ER, it is predicted that lack (or decreased) NPCL1 activity could have a number of effects on cellular homeostasis. Such effects include, (i) limiting the amount of nutrients (lipids, proteins, sugars) that become available for cellular processes, (ii) altering signaling cascades that tell the cell to behave as if nutrients are plentiful, and (iii) stimulating a limited nutrient response.

### Estimating Life Expectancy

The temporal extension of life resulting from administration of NPC1L1 antagonists to individuals can be determined by direct comparison with individuals in similar health who are not being administered the NPC1L1 antagonist, via controlled clinical studies. However, other statistics-based methods can be used to determine the prolonged life of an individual being administered an NPC1L1 antagonist.

For example, the expected lifespan of an individual, either from birth or from various ages, can be determined using standard statistical means, taking into consideration variables such as demographics, gender, ethnicity, age, genetic risk factors, socioeconomic status, pre-existing conditions and overall health. In the United States, estimates (using life tables based on post-censal estimates of the U.S. population) are provided by United States Life Expectancy Estimates. National Center for Health Statistics, National Vital Statistics Reports of the United States (several years). See Arias E. United States life tables, 2002. National vital statistics reports; vol 53 no 6. Hyattsville, Md.: National Center for Health Statistics. 2004. See also Hoyert, D L. Final data for 1999. National vital statistics reports; vol. 49 no 8. Hyattsville, Md.: NCHS, 2001.

A method for determining terms of years left until death or in proportion to the expanding lifespan, i.e., the median age of the population standardized for expected remaining years of life, is described in Sanderson et al., Nature. 2005 Jun. 9; 435(7043):811-3. Additional methods for estimating life expectancy are those used by, e.g., insurance companies, and include those described in Anderson et al., J Insur Med. 2005; 37(1):35-41. In addition, methods for estimating life expectancy in smaller localities are describe in Eayres et al., J Epidemiol Community Health. 2004; 58(3):243-9.

Models for estimating life expectancy in individuals having chronic diseases are described in Strauss et al., J Insur Med. 2005; 37(1):20-34. According to one estimate, the life expectancy at birth is increased by between 3 months and 6.5 years if cancer mortality was eliminated, and between 5 months and 7.5 years in the case of heart disease (Somerville et al., J Insur Med. 2005; 37(1):13-9).

For international life expectancies, the Population Reference Bureau publishes life expectancy rates for most countries in its World Population Data Sheet 2004.

Surrogate biomarkers of aging. In addition to the foregoing methods for calculating life expectancy, surrogate biomarkers can be used to calculate how much younger physically one is compared to their calendar age. Such biomarkers include, but are not limited to, free radical byproducts which can be measured in urine (M. Reilly et al., Circulation. 1996; 94:19-25); 8-hydroxy-2′-deoxyguanosine (8-OHdG), as another urinary a measure of oxidative stress (Miwa et al., Biofactors. 2004; 22(1-4):249-53); platelet MAO-B and erythrocyte Cu/Zn-superoxide dismutase (Grunblatt et al., Neurobiol Aging. 2005; 26(4):429-38); neurological biomarkers of cell senescence (Wu et al., Neurochem Int. 2005; 46(7):565-74); inflammatory markers such as kidney myeloperoxidase, vascular cell adhesion molecule (VCAM-1), and dityrosine (shown to be decreased in CR rats who live longer (Son et al., Free Radic Res. 2005; 39(3):283-9); other markers of inflammation such as decreases in the size of the naive CD8 positive T-cell pool with an increase in the number of interferon gamma-producing CD8 positive effector T cells (Almanzar et al., J Virol. 2005; 79(6):3675-83); increased production of autoantibodies (Fresca et al., Crit Rev Immunol. 2004; 24(5):297-320); accumulation of dolichol in tissues (Parentini et al., J Gerontol A Biol Sci Med Sci. 2005; 60(1):39-43); lipofuscin accumulation (Fonesca et al., Neurobiol Aging. 2005; 26(1):69-76); levels tumor suppressor molecules such as p16^{INK4a} and Arf, which are principal mediators of cellular senescence (Krishnamurthy et al., J Clin Invest. 2004; 114(9):1299-307); protein nitration and “browning” (DelMoral et al., Microsc Res Tech. 2004; 1; 64(4):304-11; Baynes et al., Biogerontology. 2000; 1(3):235-46); serum levels of serum levels of urea, creatinine, urate and sodium (Bathum et al., Clin Chim Acta. 2004; 349(1-2):143-50); the presence of neuroactive steroids such as pregnenolone (Vallee et al., J Steroid Biochem Mol Biol. 2003; 85(2-5):329-35); calcium regulation and gene expression (Toescu et al., Trends Neurosci. 2004; 27(10):614-20); markers of stress-induced and age-induced cell senescence (Toussaint et al., Biogerontology. 2002; 3(1-2):13-7); telomere length (Baird et al., Ann NY Acad Sci. 2004; 1019:265-8); other biomarkers (Ferrucci et al, J Endocrinol Invest. 2002; 25(10 Suppl):10-5); and the metabolic effects on e.g., insulin sensitivity, slightly decreased body temperature and initial energy expenditure, and better glucose tolerance (Roth et al., Eur J Clin Nutr. 2000; 54 Suppl 3:S15-20). Genetic biomarkers such as those described herein, and others involving e.g., genes involved in the regulation of DNA repair and nuclear structure, genes affecting endocrine signaling, stress responses, metabolism, and genes that affect telomere length, as reviewed in the following are also contemplated: Browner et al., Am J Med. 2004; 117(11):851-60; Kenyon, Cell. 2005; 120(4):449-60; and Park et al., Ageing Res Rev. 2005; 4(1):55-65.

### NPC1L1 Antagonists

NPC1L1 antagonists can include, without limitation, compounds: nucleic acids, peptides, peptide mimetics and antibodies. In one specific embodiment of the invention, the NPC1L1 antagonist is ezetimibe (Zetia®). In other embodiments, the NPC1L1 antagonist is an NPC1L1 antisense, ribozyme, or inhibitory RNA molecule.

### NPC1L1 Antibodies

Antibodies against NPC1L1 are described in published U.S. patent application 2004/0161838, to Altmann et al., hereby incorporated by reference in its entirety. Such antibodies are designated A0715, A0716, A0717, A0718, A0867, A0868, A1801 or A1802. Additional commercially available antibodies include NPC1L1 rabbit polyclonal antibodies (Novus Biologicals, Littleton, Colo., Cat #BC-400 NPC3).

### NPC1L1 Nucleic Acids

the NPC1L1-encoding nucleic acid molecules of the can be used to inhibit the expression of NPC1L1 genes (e.g., by inhibiting transcription, splicing, transport, or translation or by promoting degradation of corresponding mRNAs). Specifically, the nucleic acid molecules of the invention can be used to “knock down” or “knock out” the expression of the NPC1L1 genes in a cell or tissue (e.g., in vivo or in cultured cells in vitro for administration in vivo) by using their sequences to design antisense oligonucleotides, RNA interference (RNAi) molecules, ribozymes, nucleic acid molecules to be used in triplex helix formation, etc. Preferred methods to inhibit gene expression are described below.

### RNA Interference (RNAi)

RNA interference (RNAi) is a process of sequence-specific post-transcriptional gene silencing by which double stranded RNA (dsRNA) homologous to a target locus can specifically inactivate gene function in plants, fungi, invertebrates, and vertebrates, including mammals (Hammond et al., Nature Genet. 2001; 2: 110-119; Sharp, Genes Dev. 1999; 13: 139-141). This dsRNA-induced gene silencing is mediated by short double-stranded small interfering RNAs (siRNAs) generated from longer dsRNAs by ribonuclease III cleavage (Bernstein et al., Nature 2001; 409: 363-366 and Elbashir et al., Genes Dev. 2001; 15: 188-200). RNAi-mediated gene silencing is thought to occur via sequence-specific mRNA degradation, where sequence specificity is determined by the interaction of an siRNA with its complementary sequence within a target mRNA (see, e.g., Tuschl, Chem. Biochem. 2001; 2: 239-245).

For mammalian systems, RNAi commonly involves the use of dsRNAs that are greater than 500 bp; however, it can also be activated by introduction of either siRNAs (Elbashir, et al., Nature 2001; 411: 494-498) or short hairpin RNAs (shRNAs) bearing a fold back stem-loop structure (Paddison et al., Genes Dev. 2002; 16: 948-958; Sui et al., Proc. Natl. Acad. Sci. USA 2002; 99: 5515-5520; Brummelkamp et al., Science 2002; 296: 550-553; Paul et al., Nature Biotechnol. 2002; 20: 505-508).

The NPC1L1 siRNAs to be used in the methods of the present invention are, in one embodiment, short double stranded nucleic acid duplexes comprising annealed complementary single stranded nucleic acid molecules. In some embodiments, the siRNAs are short dsRNAs comprising annealed complementary single strand RNAs. However, the invention also encompasses embodiments in which the siRNAs comprise an annealed RNA:DNA duplex, wherein the sense strand of the duplex is a DNA molecule and the antisense strand of the duplex is a RNA molecule.

Preferably, each single stranded nucleic acid molecule of the siRNA duplex is of from about 19 nucleotides to about 27 nucleotides in length. In preferred embodiments, duplexed siRNAs have a 2 or 3 nucleotide 3′ overhang on each strand of the duplex. In preferred embodiments, siRNAs have 5′-phosphate and 3′-hydroxyl groups.

The RNAi molecules to be used in the methods of the present invention comprise nucleic acid sequences that are complementary to the nucleic acid sequence of a portion of the target locus. In certain embodiments, the portion of the target locus to which the RNAi probe is complementary is at least about 15 nucleotides in length. In preferred embodiments, the portion of the target locus to which the RNAi probe is complementary is at least about 19 nucleotides in length. The target locus to which an RNAi probe is complementary may represent a transcribed portion of the NPC1L1 gene or an untranscribed portion of the NPC1L1 gene (e.g., intergenic regions, repeat elements, etc.).

The RNAi molecules may include one or more modifications, either to the phosphate-sugar backbone or to the nucleoside. For example, the phosphodiester linkages of natural RNA may be modified to include at least one heteroatom other than oxygen, such as nitrogen or sulfur. In this case, for example, the phosphodiester linkage may be replaced by a phosphothioester linkage. Similarly, bases may be modified to block the activity of adenosine deaminase. Where the RNAi molecule is produced synthetically, or by in vitro transcription, a modified ribonucleoside may be introduced during synthesis or transcription.

According to the present invention, siRNAs may be introduced to a target cell as an annealed duplex siRNA, or as single stranded sense and anti-sense nucleic acid sequences that, once within the target cell, anneal to form the siRNA duplex. Alternatively, the sense and anti-sense strands of the siRNA may be encoded on an expression construct that is introduced to the target cell. Upon expression within the target cell, the transcribed sense and antisense strands may anneal to reconstitute the siRNA.

The shRNAs to be used in the methods of the present invention comprise a single stranded “loop” region connecting complementary inverted repeat sequences that anneal to form a double stranded “stem” region. Structural considerations for shRNA design are discussed, for example, in McManus et al., RNA 2002; 8: 842-850. In certain embodiments the shRNA may be a portion of a larger RNA molecule, e.g., as part of a larger RNA that also contains U6 RNA sequences (Paul et al., supra).

In preferred embodiments, the loop of the shRNA is from about 1 to about 9 nucleotides in length. In preferred embodiments the double stranded stem of the shRNA is from about 19 to about 33 base pairs in length. In preferred embodiments, the 3′ end of the shRNA stem has a 3′ overhang. In particularly preferred embodiments, the 3′ overhang of the shRNA stem is from 1 to about 4 nucleotides in length. In preferred embodiments, shRNAs have 5′-phosphate and 3′-hydroxyl groups.

Although the RNAi molecules useful according to the invention preferably contain nucleotide sequences that are fully complementary to a portion of the target locus, 100% sequence complementarity between the RNAi probe and the target locus is not required to practice the invention.

RNA molecules useful for RNAi may be chemically synthesized, for example using appropriately protected ribonucleoside phosphoramidites and a conventional DNA/RNA synthesizer. RNAs produced by such methodologies tend to be highly pure and to anneal efficiently to form siRNA duplexes or shRNA hairpin stem-loop structures. Following chemical synthesis, single stranded RNA molecules are deprotected, annealed to form siRNAs or shRNAs, and purified (e.g., by gel electrophoresis or HPLC).

Alternatively, standard procedures may used for in vitro transcription of RNA from DNA templates carrying RNA polymerase promoter sequences (e.g., T7 or SP6 RNA polymerase promoter sequences). Efficient in vitro protocols for preparation of siRNAs using T7 RNA polymerase have been described (Done and Picard, Nucleic Acids Res. 2002; 30: e46; and Yu et al., Proc. Natl. Acad. Sci. USA. 2002; 99: 6047-6052). Similarly, an efficient in vitro protocol for preparation of shRNAs using T7 RNA polymerase has been described (Yu et al., supra). The sense and antisense transcripts may be synthesized in two independent reactions and annealed later, or may be synthesized simultaneously in a single reaction.

RNAi molecules may be formed within a cell by transcription of RNA from an expression construct introduced into the cell. For example, both a protocol and an expression construct for in vivo expression of siRNAs are described in Yu et al., supra. Similarly, protocols and expression constructs for in vivo expression of shRNAs have been described (Brummelkamp et al., supra; Sui et al., supra; Yu et al., supra; McManus et al., supra; Paul et al., supra).

The expression constructs for in vivo production of RNAi molecules comprise RNAi encoding sequences operably linked to elements necessary for the proper transcription of the RNAi encoding sequence(s), including promoter elements and transcription termination signals. Preferred promoters for use in such expression constructs include the polymerase-III HI-RNA promoter (see, e.g., Brummelkamp et al., supra) and the U6 polymerase-III promoter (see, e.g., Sui et al., supra; Paul, et al. supra; and Yu et al., supra). The RNAi expression constructs can further comprise vector sequences that facilitate the cloning of the expression constructs. Standard vectors that maybe used in practicing the current invention are known in the art (e.g., pSilencer 2.0-U6 vector, Ambion Inc., Austin, Tex.).

### Other NCP1L1 Antagonists

The following NCP1L1 inhibitors have been identified (see Example 2) which are contemplated for use in the methods of the present invention:

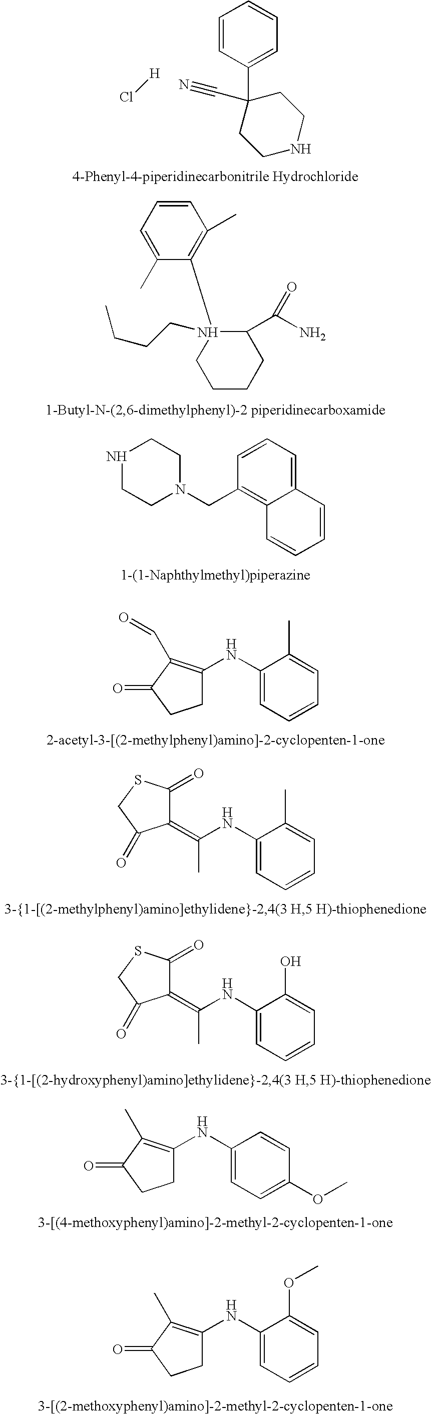

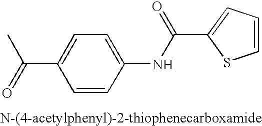

Other NPC1L1 antagonists were described in provisional patent application 60/592,592, filed on Jul. 30, 2004, and WO 2006/015365 (corresponding to International Patent Application No. PCT/US2005/027579, filed Aug. 1, 2005) herein incorporated by reference. Such inhibitors include the following:

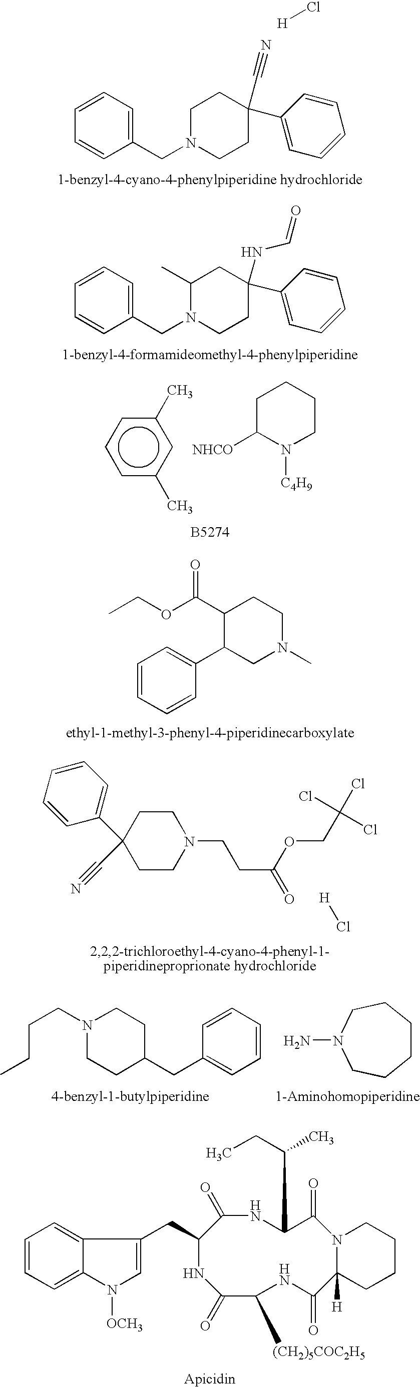

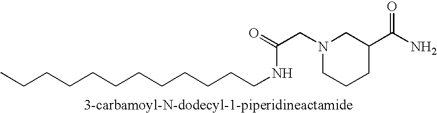

Also contemplated by the present invention are agents that inhibit NPC1L1 at the transcriptional level, such as agents which bind to an inhibit the NCP1L1 promoter, or agents which inhibit specific transcription factors which regulate the NPC1L1 promoter or enhancer region.

### Screening for NPC1L1 Antagonists

Screening to identify other candidate NPC1L1 antagonists can be achieved using routine methods in the art. Such screening methods are described in published U.S. patent applications 2004/0161838, to Altmann et al. Other screening methods are disclosed in provisional patent application Ser. No. 60/592,592, filed Jul. 30, 2004, and WO 2006/015365 (corresponding to International Patent Application No. PCT/US2005/027579, filed Aug. 1, 2005). In addition, any screening method known and used in the pertinent field can be employed to identify candidate or lead agents that are effective NPC1L1 antagonists.

Compound libraries. Collections of compounds that can be used for screening for NPC1L1 antagonists include but are not limited to those provided the following: ASDI Biosciences (Newark, Del.); Tim-Tec (Newark, Del.); Sigma Aldrich (St. Louis, Mo.); Analyticon Discovery (Germany); Comgenex (Hungary); MDPI (Switzerland); Moscow MedChem Labs (Moscow); Tripos (Missouri); Biomol (Plymouth Meeting, Pa.); Stanford High Throughput Bioscience Center (HTBC; Stanford, Calif.); ICBB Longwood Collection (Harvard; Cambridge Mass.). Additional compound libraries include Bionet 1 (4,800 compounds); Bionet 2 (1,700 compounds); CEREP (4,800 compounds); Chem Bridge DiverSet E (16,320 compounds); ChemBridge Microformat (50,000 compounds); ChemDiv1 (Combilab and International) (28,864 compounds); ChemDiv 2 (8,560 compounds); Commercial Diversity Set 1 (5,056 compounds); Enamine 1 (6,004 compounds); I.F. Lab 1 (6,543 compounds); I.F. Lab 2 (292 compounds); Maybridge 1 (8,800 compounds); Maybridge 2 (704 compounds); Maybridge 3 (7,639 compounds); Peakdale 1 (2,816 compounds); Peakdale 2 (352 compounds); Mixed Commercial Plate 1 (352 compounds); Mixed Commercial Plate 2 (320 compounds); Mixed Commercial Plate 3 (251 compounds); Mixed Commercial Plate 4 (331 compounds); Structural Diversity Set, version 1 (1,991 compounds); Structural Diversity Set, version 2 (1,986 compounds); Mechanistic Diversity Set (879 compounds); Open Collection 1 (90,000 compounds); Open Collection 2 (10,000 compounds). Known bioactives collections include NINDS Custom Collection (1,040 compounds); ICCB Bioactives 1 (489 compounds); SpecPlus Collection (960 compounds); BIOMOL ICCB Known Bioactives (480 compounds). Natural products collections include ICBG Fungal Extracts 1 (851 wells); NCI Marine Extracts (352 wells); Aqueous fractions—NCI Plant and Fungal Extracts (2,112 wells); Organic fractions—NCI Plant and Fungal Extracts (1,408 wells); Philippines Plant Extracts 1 (200 wells); Philippines Plant Extracts 2 (648 wells); and Starr Foundation Extracts 1 (1025 wells).

### Antisense Nucleic Acids

In a specific embodiment, to achieve inhibition of expression of a NPC1L1 gene, the nucleic acid molecules of the invention can be used to design antisense oligonucleotides. An antisense oligonucleotide is typically 18 to 25 bases in length (but can be as short as 13 bases in length) and is designed to bind to a selected NPC1L1mRNA. This binding prevents expression of that specific NPC1L1 protein. The antisense oligonucleotides of the invention comprise at least 6 nucleotides and preferably comprise from 6 to about 50 nucleotides. In specific aspects, the antisense oligonucleotides comprise at least 10 nucleotides, at least 15 nucleotides, at least 25, at least 30, at least 100 nucleotides, or at least 200 nucleotides.

The antisense nucleic acid oligonucleotides of the invention comprise sequences complementary to at least a portion of the corresponding NPC1L1 mRNA. However, 100% sequence complementarity is not required so long as formation of a stable duplex (for single stranded antisense oligonucleotides) or triplex (for double stranded antisense oligonucleotides) can be achieved. The ability to hybridize will depend on both the degree of complementarity and the length of the antisense oligonucleotides. Generally, the longer the antisense oligonucleotide, the more base mismatches with the corresponding mRNA can be tolerated. One skilled in the art can ascertain a tolerable degree of mismatch by use of standard procedures to determine the melting point of the hybridized complex.

The antisense oligonucleotides can be DNA or RNA or chimeric mixtures, or derivatives or modified versions thereof, and can be single-stranded or double-stranded. The antisense oligonucleotides can be modified at the base moiety, sugar moiety, or phosphate backbone, or a combination thereof. For example, a NPC1L1-specific antisense oligonucleotide can comprise at least one modified base moiety selected from a group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the NPC1L1-specific antisense oligonucleotide comprises at least one modified sugar moiety, e.g., a sugar moiety selected from arabinose, 2-fluoroarabinose, xylulose, and hexose.

In yet another embodiment, the NPC1L1-specific antisense oligonucleotide comprises at least one modified phosphate backbone selected from a phosphorothioate, a phosphorodithioate, a phosphoramidothioate, a phosphoramidate, a phosphordiamidate, a methylphosphonate, an alkyl phosphotriester, and a formacetal or analog thereof.

The antisense oligonucleotide can include other appending groups such as peptides, or agents facilitating transport across the cell membrane (see, e.g., Letsinger et al., Proc. Natl. Acad. Sci. USA. 1989; 86: 6553-6556; Lemaitre et al., Proc. Natl. Acad. Sci. USA 1987; 84: 648-652; PCT Publication No. WO 88/09810) or blood-brain barrier (see, e.g., PCT Publication No. WO 89/10134), hybridization-triggered cleavage agents (see, e.g., Krol et al., BioTechniques: 1988; 6: 958-976), intercalating agents (see, e.g., Zon, Pharm. Res. 1988; 5: 539-549), etc.

In another embodiment, the antisense oligonucleotide can include α-anomeric oligonucleotides. An α-anomeric oligonucleotide forms specific double-stranded hybrids with complementary RNA in which, contrary to the usual β-units, the strands run parallel to each other (Gautier et al., Nucl. Acids Res. 1987; 15: 6625-6641).

In yet another embodiment, the antisense oligonucleotide can be a morpholino antisense oligonucleotide (i.e., an oligonucleotide in which the bases are linked to 6-membered morpholine rings, which are connected to other morpholine-linked bases via non-ionic phosphorodiamidate intersubunit linkages). Morpholino oligonucleotides are resistant to nucleases and act by sterically blocking transcription of the target mRNA.

Similar to the above-described RNAi molecules, the antisense oligonucleotides of the invention can be synthesized by standard methods known in the art, e.g., by use of an automated synthesizer. Antisense nucleic acid oligonucleotides of the invention can also be produced intracellularly by transcription from an exogenous sequence.

The present invention thus provides a method for inhibiting the expression of a NPC1L1 gene in a eukaryotic, preferably mammalian, and more preferably rat, mouse or human cell, comprising providing the cell with an effective amount of a NPC1L1-inhibiting antisenseoligonucleotide.

### Ribozyme and Triple Helix Inhibition

In another embodiment, the expression of NPC1L1 genes of the present invention can be inhibited by ribozymes designed based on the nucleotide sequence thereof. Ribozyme molecules catalytically cleave mRNA transcripts and can be used to prevent expression of the gene product. Ribozymes are enzymatic RNA molecules capable of catalyzing the sequence-specific cleavage of RNA (for a review, see Rossi, Current Biology 1994; 4: 469-471).

Ribozymes can be prepared by any method known in the art for the synthesis of DNA and RNA molecules, as discussed above. Ribozyme technology is described further in Intracellular Ribozyme Applications: Principals and Protocols, Rossi and Couture eds., Horizon Scientific Press, 1999.

Similarly to NPC1L1-specific RNAi, antisense oligonucleotides, and ribozymes, triple helix molecules of the invention can be prepared by any method known in the art. These include techniques for chemically synthesizing oligodeoxyribonucleotides and oligoribonucleotides such as, e.g., solid phase phosphoramidite chemical synthesis. Alternatively, RNA molecules can be generated by in vitro or in vivo transcription of DNA sequences “encoding” the particular RNA molecule. Such DNA sequences can be incorporated into a wide variety of vectors that incorporate suitable RNA polymerase promoters such as the T7 or SP6 polymerase promoters.

### Gene Therapy Using NPC1L1 Antagonists

In one embodiment, the present invention contemplates inhibiting NPC1L1 in vivo, by administering to an individual the above-disclosed NPC1L1 inhibitor nucleic acid, i.e., gene therapy.

Any of the methods for gene therapy available in the art can be used according to the present invention. Exemplary methods are described below. For general reviews of the methods of gene therapy, see Goldspiel et al., Clinical Pharmacy. 1993, 12:488-505; Wu and Wu, Biotherapy. 1991, 3:87-95; Tolstoshev, Ann. Rev. Pharmacol. Toxicol. 1993, 32:573-596; Mulligan, Science. 1993, 260:926-932; and Morgan and Anderson, Ann. Rev. Biochem. 1993, 62:191-217; May, TIBTECH. 1993, 11:155-215. Methods commonly known in the art of recombinant DNA technology that can be used are described in Ausubel et al., (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al., (eds.), 1994, Current Protocols in Human Genetics, John Wiley & Sons, NY; Colosimo et al., Biotechniques 2000; 29(2):314-8, 320-2, 324; among others.

The gene to be administered for the methods of the present invention can be isolated and purified using ordinary molecular biology, microbiology, and recombinant DNA techniques within the skill of the art. For example, nucleic acids encoding the target protein can be isolated using recombinant DNA expression as described in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. ÊHiggins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. ÊPerbal, A Practical Guide To Molecular Cloning (1984); F. M. Ausubel et al. (eds.), Current Protocols in Molecular Biology, John Wiley & Sons, Inc. (1994); Ausubel et al. eds., Current Protocols in Molecular Biology, John Wiley and Sons, Inc. 1994; Sambrook et al. (2001) Molecular Cloning: A Laboratory Manual. 3^{rd} ed. Cold Spring Harbor Laboratory Press: Cold Spring Harbor, N.Y. (herein “Sambrook et al., 2001”); Ausubel et al. eds. (2006) Current Protocols in Molecular Biology. John Wiley and Sons, Inc.: Hoboken, N.J.; Bonifacino et al. eds. (2006) Current Protocols in Cell Biology. John Wiley and Sons, Inc.: Hoboken, N.J.; Coligan et al. eds. (2006) Current Protocols in Immunology, John Wiley and Sons, Inc.: Hoboken, N.J.; Coico et al. eds. (2006) Current Protocols in Microbiology, John Wiley and Sons, Inc.: Hoboken, N.J.; Coligan et al. eds. (2006) Current Protocols in Protein Science, John Wiley and Sons, Inc.: Hoboken, N.J.; Enna et al. eds. (2006) Current Protocols in Pharmacology John Wiley and Sons, Inc.: Hoboken, N.J.; Hames et al. eds. (1999) Protein Expression: A Practical Approach. Oxford University Press: Oxford; Freshney (2000) Culture of Animal Cells: A Manual of Basic Technique. 4^{th} ed. Wiley-Liss; among others (the Current Protocols listed above are updated several times every year). The nucleic acid encoding the protein may be full-length or truncated, so long as the gene encodes a biologically active protein.

The identified and isolated gene can then be inserted into an appropriate cloning vector. Vectors suitable for gene therapy include viruses, such as adenoviruses, adeno-associated virus (AAV), vaccinia, herpesviruses, baculoviruses and retroviruses, parvovirus, lentivirus, bacteriophages, cosmids, plasmids, fungal vectors, and other recombination vehicles typically used in the art which have been described for expression in a variety of eukaryotic and prokaryotic hosts, and may be used for gene therapy as well as for simple protein expression.

In a preferred embodiment, the vector is a viral vector. Viral vectors, especially adenoviral vectors can be complexed with a cationic amphiphile, such as a cationic lipid, polyL-lysine (PLL), and diethylaminoethyldextran (DELAE-dextran), which provide increased efficiency of viral infection of target cells (See, e.g., PCT/US97/21496 filed Nov. 20, 1997, published as WO 1998/022144, incorporated herein by reference). Preferred viral vectors for use in the present invention include vectors derived from vaccinia, herpesvirus, AAV and retroviruses. In particular, herpesviruses, especially herpes simplex virus (HSV), such as those disclosed in U.S. Pat. No. 5,672,344, the disclosure of which is incorporated herein by reference, are particularly useful for delivery of a transgene to a neuronal cell. AAV vectors, such as those disclosed in U.S. Pat. Nos. 5,139,941, 5,252,479 and 5,753,500 and PCT publication WO 97/09441, the disclosures of which are incorporated herein, are also useful since these vectors integrate into host chromosomes, with a minimal need for repeat administration of vector. For a review of viral vectors in gene therapy, see Mah et al., Clin. Pharmacokinet. 2002; 41(12):901-11; Scott et al., Neuronmuscul. Disord. 2002; 12 Suppl 1:S23-9. In addition, see U.S. Pat. No. 5,670,488.

The coding sequences of the gene to be delivered are operably linked to expression control sequences, e.g., a promoter that directs expression of the gene. As used herein, the phrase “operatively linked” refers to the functional relationship of a polynucleotide/gene with regulatory and effector sequences of nucleotides, such as promoters, enhancers, transcriptional and translational stop sites, and other signal sequences. For example, operative linkage of a nucleic acid to a promoter refers to the physical and functional relationship between the polynucleotide and the promoter such that transcription of DNA is initiated from the promoter by an RNA polymerase that specifically recognizes and binds to the promoter, and wherein the promoter directs the transcription of RNA from the polynucleotide.

In one specific embodiment, a vector is used in which the coding sequences and any other desired sequences are flanked by regions that promote homologous recombination at a desired site in the genome, thus providing for expression of the construct from a nucleic acid molecule that has integrated into the genome (Koller and Smithies, Próc. Natl. Acad. Sci. USA. 1989, 86:8932-8935; Zijlstra et al., Nature. 1989, 342:435-438; U.S. Pat. No. 6,244,113 to Zarling et al.; and U.S. Pat. No. 6,200,812 to Pati et al.)

Delivery of the vector into a patient may be either direct, in which case the patient is directly exposed to the vector or a delivery complex, or indirect, in which case, cells are first transformed with the vector in vitro, then transplanted into the patient. These two approaches are known, respectively, as in vivo and ex vivo gene therapy.

Direct transfer. In a specific embodiment, the vector with the inhibitor NPC1L1 nucleic acid is directly administered in vivo, where it enters the cells of the organism and mediates expression of the gene. This can be accomplished by any of numerous methods known in the art and discussed above, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell-surface receptors or transfecting agents, encapsulation in biopolymers (e.g., poly-β-1-64-N-acetylglucosamine polysaccharide; see U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem. 1987, 62:4429-4432), etc. In another embodiment, a nucleic acid-ligand complex can be formed in which the ligand comprises a fusogenic viral peptide to disrupt endosomes, allowing the nucleic acid to avoid lysosomal degradation, or cationic 12-mer peptides, e.g., derived from antennapedia, that can be used to transfer therapeutic DNA into cells (Mi et al., Mol. Therapy. 2000, 2:339-47).

In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). Recently, a technique referred to as magnetofection has been used to deliver vectors to mammals. This technique associates the vectors with superparamagnetic nanoparticles for delivery under the influence of magnetic fields. This application reduces the delivery time and enhances vector efficacy (Scherer et al., Gene Therapy. 2002; 9:102-9). Additional targeting and delivery methodologies are contemplated in the description of the vectors, below.

In a specific embodiment, the nucleic acid can be administered using a lipid carrier. Lipid carriers can be associated with naked nucleic acids (e.g., plasmid DNA) to facilitate passage through cellular membranes. Cationic, anionic, or neutral lipids can be used for this purpose. However, cationic lipids are preferred because they have been shown to associate better with DNA which, generally, has a negative charge. Cationic lipids have also been shown to mediate intracellular delivery of plasmid DNA (Feigner and Ringold, Nature. 1989; 337:387). Intravenous injection of cationic lipid-plasmid complexes into mice has been shown to result in expression of the DNA in lung (Brigham et al., Am. J. Med. Sci. 1989; 298:278). See also, Osaka et al., J. Pharm. Sci. 1996; 85(6):612-618; San et al., Human Gene Therapy 1993; 4:781-788; Senior et al., Biochemica et Biophysica Acta. 1991; 1070:173-179); Kabanov and Kabanov, Bioconjugate Chem. 1995; 6:7-20; Liu et al., Pharm Res. 1996; 13(10):1501-63; Liu et al. Pharm Res. 1996; 13(11):1642-6; Liu et al., Pharm Res. 1996; 13(12):1856-60; Remy et al., Bioconjugate Chem. 1994; 5:647-654; Behr, J-P., Bioconjugate Chem 1994; 5:382-389; Wyman et al., Biochem. 1997; 36:3008-3017; U.S. Pat. No. 5,939,401 to Marshall et al; U.S. Pat. No. 6,331,524 to Scheule et al.

Representative cationic lipids include those disclosed, for example, in U.S. Pat. No. 5,283,185; and e.g., U.S. Pat. No. 5,767,099, the disclosures of which are incorporated herein by reference. In a preferred embodiment, the cationic lipid is N₄-spermine cholesteryl carbamate (GL-67) disclosed in U.S. Pat. No. 5,767,099. Additional preferred lipids include N₄-spermidine cholestryl carbamate (GL-53) and 1-(N₄-spermine)-2,3-dilaurylglycerol carbamate (GL-89)

Preferably, for in vivo administration of viral vectors, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors. In that regard, it is advantageous to employ a viral vector that is engineered to express a minimal number of antigens.

Recently, one group described a new peptide-based gene delivery system, MPG, which forms stable noncovalent complexes with several nucleic acids (plasmid DNA, oligonucleotides) and promotes their delivery into a large panel of cell lines without the need for prior chemical covalent coupling (Simeoni et al., Methods Mol Biol. 2005; 309:251-60). Other recent methods for delivery of nucleic acids include

Indirect transfer. Somatic cells may be engineered ex vivo with a construct encoding a wild-type protein using any of the methods described above, and re-implanted into an individual. This method is described generally in WO 93/09222 to Selden et al. In addition, this technology is used in Cell Based Delivery's proprietary ImPACT technology, described in Payumo et al., Clin. Orthopaed. and Related Res. 2002; 403S: S228-S242. In such a gene therapy system, somatic cells (e.g., fibroblasts, hepatocytes, or endothelial cells) are removed from the patient, cultured in vitro, transfected with the gene(s) of therapeutic interest, characterized, and reintroduced into the patient. Both primary cells (derived from an individual or tissue and engineered prior to passaging), and secondary cells (passaged in vitro prior to introduction in vivo) can be used, as well as immortalized cell lines known in the art. Somatic cells useful for the methods of the present invention include but are not limited to somatic cells, such as fibroblasts, keratinocytes, epithelial cells, endothelial cells, glial cells, neural cells, formed elements of the blood, muscle cells, other somatic cells that can be cultured, and somatic cell precursors. In a preferred embodiment, the cells are fibroblasts or mesenchymal stem cells.

Nucleic acid constructs, which include the exogenous gene and, optionally, nucleic acids encoding a selectable marker, along with additional sequences necessary for expression of the exogenous gene in recipient primary or secondary cells, are used to transfect primary or secondary cells in which the encoded product is to be produced. Such constructs include but are not limited to infectious vectors, such as retroviral, herpes, adenovirus, adenovirus-associated, mumps and poliovirus vectors, can be used for this purpose.

Transdermal delivery is especially suited for indirect transfer using cell types of the epidermis including keratinocytes, melanocytes, and dendritic cells (Pfutzner et al., Expert Opin. Investig. Drugs. 2000; 9:2069-83).

Mesenchymal stem cells (MSCs) are non-blood-producing stein cells produced in the bone marrow. MSCs can be made to differentiate and proliferate into specialized non-blood tissues. Stem cells transfected with retroviruses are good candidates for the therapy due to their capacity for self-renewal. This ability precludes repetitive administration of the gene therapy. Another advantage is that if the injected stem cells reach the target organ and then differentiate, they can replace the damaged or malformed cells at the organ.

### Formulations and Administration

An NPC1L1 antagonist useful for the method of the present invention is advantageously formulated in a pharmaceutical composition with a pharmaceutically acceptable carrier. The candidate compound may be designated as an active ingredient or therapeutic agent for prolonging life.

The NPC1L1 antagonist can be administered in a form suitable for any route of administration, including e.g., orally in the form tablets or capsules or liquid, or in sterile aqueous solution for injection. When the NPC1L1 antagonist is formulated for oral administration, the tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art.

Liquid preparations for oral administration may take the form of, for example, solutions, syrups or suspensions, or they may be presented as a dry product for constitution with water or another suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled or sustained release of the specific pharmacological chaperone.

The pharmaceutical formulations of the NPC1L1 antagonist suitable for parenteral/injectable use generally include sterile aqueous solutions (where water soluble), or dispersions and sterile powders for the extemporaneous preparation of sterile injectable solutions or dispersion. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alcohol, sorbic acid, and the like. In many cases, it will be reasonable to include isotonic agents, for example, sugars or sodium chloride. Prolonged absorption of the injectable compositions can be brought about by the use in the compositions of agents delaying absorption, for example, aluminum monosterate and gelatin.

Sterile injectable solutions are prepared by incorporating the specific pharmacological chaperone in the required amount in the appropriate solvent with various of the other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The formulation can contain an excipient. Pharmaceutically acceptable excipients which may be included in the formulation are buffers such as citrate buffer, phosphate buffer, acetate buffer, and bicarbonate buffer, amino acids, urea, alcohols, ascorbic acid, phospholipids; proteins, such as serum albumin, collagen, and gelatin; salts such as EDTA or EGTA, and sodium chloride; liposomes; polyvinylpyrollidone; sugars, such as dextran, mannitol, sorbitol, and glycerol; propylene glycol and polyethylene glycol (e.g., PEG-4000, PEG-6000); glycerol; glycine or other amino acids; and lipids. Buffer systems for use with the formulations include citrate; acetate; bicarbonate; and phosphate buffers. Phosphate buffer is a preferred embodiment.

The formulation can also contain a non-ionic detergent. Preferred non-ionic detergents include Polysorbate 20, Polysorbate 80, Triton X-100, Triton X-114, Nonidet P-40, Octyl α-glucoside, Octyl β-glucoside, Brij 35, Pluronic, and Tween 20.

### Administration

The route of administration of the NPC1L1 antagonist may be oral (preferably) or parenteral, including intravenous, subcutaneous, intra-arterial, intraperitoneal, ophthalmic, intramuscular, buccal, rectal, vaginal, intraorbital, intracerebral, intradermal, intracranial, intraspinal, intraventricular, intrathecal, intracisternal, intracapsular, intrapulmonary, intranasal, transmucosal, transdermal, or via inhalation.

Administration of the above-described parenteral formulations of the NPC1L1 antagonist may be by periodic injections of a bolus of the preparation, or may be administered by intravenous or intraperitoneal administration from a reservoir which is external (e.g., an i.v. bag) or internal (e.g., a bioerodable implant). See, e.g., U.S. Pat. Nos. 4,407,957 and 5,798,113, each incorporated herein by reference. Intrapulmonary delivery methods and apparatus are described, for example, in U.S. Pat. Nos. 5,654,007, 5,780,014, and 5,814,607, each incorporated herein by reference. Other useful parenteral delivery systems include ethylene-vinyl acetate copolymer particles, osmotic pumps, implantable infusion systems, pump delivery, encapsulated cell delivery, liposomal delivery, needle-delivered injection, needle-less injection, nebulizer, aeorosolizer, electroporation, and transdermal patch. Needle-less injector devices are described in U.S. Pat. Nos. 5,879,327; 5,520,639; 5,846,233 and 5,704,911, the specifications of which are herein incorporated by reference. Any of the formulations described above can be administered using these methods.

Subcutaneous injections have the advantages allowing self-administration, while also resulting in a prolonged plasma half-life as compared to intravenous administration. Furthermore, a variety of devices designed for patient convenience, such as refillable injection pens and needle-less injection devices, may be used with the formulations of the present invention as discussed herein.

### Dosage

The amount of NPC1L1 antagonist effective to prolong or extend longevity can be determined on a case-by-case basis by those skilled in the art. However, the amount must be sufficient to significantly reduce or inhibit NPC1L1 gene and/or protein expression. Other factors specific to the NPC1L1 antagonist must also be considered. Such factors include pharmacokinetics and pharmacodynamics such as half-life (t_{1/2}), peak plasma concentration (Cₘₐₓ), time to peak plasma concentration (tₘₐₓ), exposure as measured by area under the curve (AUC), and tissue distribution the NPC1L1 antagonist. Such information can be obtained using ordinary methods known in the art to determine compatible amounts required to stabilize the replacement protein, without inhibiting its activity, and thus confer a therapeutic effect.

Data obtained from cell culture assay or animal studies may be used to formulate a therapeutic dosage range for use in humans and non-human animals. The dosage of compounds used in therapeutic methods of the present invention preferably lie within a range of circulating concentrations that includes the ED₅₀ concentration (effective for 50% of the tested population) but with little or no toxicity. The particular dosage used in any treatment may vary within this range, depending upon factors such as the particular dosage form employed, the route of administration utilized, the conditions of the individual (e.g., patient), and so forth.

A therapeutically effective dose may be initially estimated from cell culture assays and formulated in animal models, to achieve a circulating concentration range that includes the IC₅₀. The IC₅₀ concentration of a compound is the concentration that achieves a half-maximal inhibition of symptoms (e.g., as determined from the cell culture assays). Appropriate dosages for use in a particular individual, for example in human patients, may then be more accurately determined using such information.

Measures of compounds in plasma may be routinely measured in an individual such as a patient by techniques such as high performance liquid chromatography (HPLC) or gas chromatography.

Toxicity and therapeutic efficacy of the composition can be determined by standard pharmaceutical procedures, for example in cell culture assays or using experimental animals to determine the LD₅₀ and the ED₅₀. The parameters LD₅₀ and ED₅₀ are well known in the art, and refer to the doses of a compound that is lethal to 50% of a population and therapeutically effective in 50% of a population, respectively. The dose ratio between toxic and therapeutic effects is referred to as the therapeutic index and may be expressed as the ratio: LD₅₀/ED₅₀. Specific pharmacological chaperones that exhibit large therapeutic indices are preferred.

Accordingly, the concentration of the NPC1L1 antagonist depends on the desired dosage and administration regimen, as discussed below. Suitable dose ranges of the active ingredient are from about 0.01 mg/kg to about 1500 mg/kg of body weight per day. For example, the recommended dose for the NPC1L1 antagonist Zetia®, for treating hypercholesterolemia, is 10 mg/day. Following oral administration, Zetia® is rapidly absorbed and extensively metabolised (>80%) to the pharmacologically active ezetimibe-glucuronide. Total Zetia® (sum of ‘parent’ ezetimibe plus ezetimibe-glucuronide) concentrations reach a maximum 1-2 hours post-administration, followed by enterohepatic recycling and slow elimination. The estimated terminal half-life of ezetimibe and ezetimibe-glucuronide is approximately 22 hours. Consistent with the elimination half-life of Zetia®, an approximate 2-fold accumulation is observed upon repeated once-daily administration.

The dose for prolonging or extending life may be lower or higher than the dose required to treat hypercholesterolemia. Effective dosages may be in the range of 0.5-20 mg/day, 5.0 to 15 mg/day, or 10 mg/day. These dosages are also contemplated for the other disclosed NPC1L1 inhibitors.

### Combination Therapy

The present invention contemplates combination drug therapy with other agents that are known for extending or prolonging life. Such agents include, but are not limited to, deprenyl, melatonin, and centrophenoxine, dehydroepiandrosterone (DHEA), synthetic human growth hormone, piracetam, vinpocetine-hydergine, procaine, centrophenoxine, phosophatidylserine, acetyl-L-carnatine, and aspirin. Combination therapy with agents that inhibit oxidative stress (e.g., antioxidants to inhibit reactive oxygen intermediates) is also contemplated. Such agents include synthetic catalytic scavengers of superoxide dismutase (SOD) (Eukaryion, Inc., Bedford, Mass.), and antioxidants including vitamins such as E, and C and beta carotene, and any thiol-specific antioxidant enzymes (M. B. Yim et al., Journal of Biochemistry. 1994; 269:1621-6).

Combination therapy with the NPC1L1 antagonists of the present invention with caloric restriction is also contemplated. Caloric restriction can be implemented either as reduced regular feeding, or as days of fasting alternating with days of free-feeding. In another embodiment, agents such as resveratrol (trans-3,5,4′-trihydroxystilbene), which have been shown to mimic effects of caloric restriction, can be combined with the NPC1L1 antagonists of the present invention. Resveratrol is a polyphenol found in the skin of red grapes and has been shown to activate the SIRT1 gene discussed above. Other agents include metformin (Glucophage®), which improves insulin resistance, hormone therapy with estrogen, progesterone, and testosterone, or agents such as ubiquinone.

In addition, combination therapy is contemplated where the individual has an underlying chronic disease or disorder, i.e., combination therapy with the NPC1L1 antagonists and the agents used to treat the underlying disorder. Such agents may include but are not limited to analgesics, anti-inflammatories, anti-arrhythimics, anti-arthritics, anti-anxiety agents, anti-cholinergics, anti-diabetics, antidepressants, anti-retrovirals, benzodiazepines, antipsychotics, beta-blockers, biguanides, calcium channel blockers, cardiac glycosides, ergot alkyloids, insulin, NSAIDS, neuroleptics, opioids, oral hypoglycemics, proton pump inhibitors, stimulants, rheumatologicals, corticosteroids, etc.

### EXAMPLES

The present invention is further described by way of the following particular examples. However, the use of such examples is illustrative only and is not intended to limit the scope or meaning of this invention or of any exemplified term. Nor is the invention limited to any particular preferred embodiment(s) described herein. Indeed, many modifications and variations of the invention will be apparent to those skilled in the art upon reading this specification, and such “equivalents” can be made without departing from the invention in spirit or scope. The invention is therefore limited only by the terms of the appended claims, along with the full scope of equivalents to which the claims are entitled.

### Example 1

### Extended Life of NPC1L1 Knock-Out Mice

### Rationale

It is well established in lower organisms that caloric restriction has the unexpected benefit of extending life span. Since NPC1L1 is a regulator of multiple lipid uptake and transport, we reasoned that it's inactivation may have an effect on extending longevity due to effects similar to caloric restriction. Since NPC1L1 appears to regulate the flow of lipids (and possibly other nutrients) from the plasma membrane (uptake) to the various cellular organelles such as Golgi and ER, we hypothesized that lack (or decreased) NPC1L1 activity could have a number of effects on cellular homeostasis: 1) limit the amount of nutrients (lipids, proteins, sugars) that become available for cellular processes, 2) alter signaling cascades that tell the cell to behave as if nutrients are plentiful, and 3) stimulate a limited nutrient response.

### Methods

NPC1L1 −/− mice were generated as described previously in provisional patent application Ser. No. 60/592,592, filed Jul. 30, 2004, and WO 2006/015365 (corresponding to International Patent Application No. PCT/US2005/027579, filed Aug. 1, 2005), and also in Davies et al., J Biol Chem. 2005; 280(13):12710-20.

### Results

There are a number of reports in the literature on the life span of male mice from the C57BK6 strain of mice used to generate the NPC1L1 −/− mice. These reports vary, with some reporting life spans of 676 days (Storer et al., J. Gerontol. 1996; 21: 404-409) for conventional conditions similar to those used in this study (e.g., mice are bred in a “clean facility,” in cages with automated food and water dispension and are checked daily by staff) 827±34 days (Goodrick et al., J. Gerontol. 1975; 30: 257-263.), and 878±10 days (Kunstyr et al., J. Gerontol. 1975; 30:157-162). Taking an average from the published data we get 793 days of life.

All of the NPC1L1 −/− mice that are now deceased lived passed this average number of days (Table 1), demonstrating increased longevity. Table 2 presents data for other mice still alive as of Jul. 29, 2006.

TABLE 1
Age in days of oldest NPC1L1 −/−mice, now deceased

| DOB | Days Old | Sex |
|---|---|---|
| Jan. 12, 2003 | 1039.00 | M |
| Jan. 12, 2003 | 1088.00 | M |
| Feb. 26, 2003 | 984.00 | M |
| Mar. 12, 2003 | 990.00 | M |
| Aug. 17, 2003 | 1012.00 | M |
| Jul. 21, 2003 | 939.00 | F |
| Feb. 08, 2004 | 837.00 | M |

TABLE 2
Age in days of oldest NPC1L1 −/−mice, living as of Jul. 29, 2006

| DOB | Days Old | Sex |
|---|---|---|
| Jun. 4, 2004 | 787 | M |
| Jun. 4, 2004 | 787 | M |
| Jun. 30, 2004 | 761 | M |
| Jun. 30, 2004 | 761 | M |
| Jun. 30, 2004 | 761 | M |
| Jun. 30, 2004 | 761 | M |
| Aug. 17, 2004 | 713 | M |
| Aug. 18, 2004 | 712 | M |
| Aug. 18, 2004 | 712 | M |
| Aug. 18, 2004 | 712 | M |
| Aug. 18, 2004 | 712 | M |
| Aug. 18, 2004 | 712 | M |
| Aug. 21, 2004 | 709 | M |
| Aug. 21, 2004 | 709 | M |
| Sep. 10, 2004 | 689 | M |
| Sep. 10, 2004 | 689 | M |
| Sep. 10, 2004 | 689 | M |
| Oct. 3, 2004 | 666 | M |
| Nov. 5, 2004 | 633 | M |
| Nov. 5, 2004 | 633 | M |
| Nov. 5, 2004 | 633 | M |
| Nov. 5, 2004 | 633 | M |
| Nov. 28, 2004 | 610 | M |
| Nov. 18, 2004 | 620 | M |
| Nov. 22, 2004 | 616 | M |
| Nov. 22, 2004 | 616 | M |
| Nov. 22, 2004 | 616 | M |
| Dec. 2, 2004 | 606 | M |
| Dec. 4, 2004 | 604 | M |
| Dec. 6, 2004 | 602 | M |
| Dec. 6, 2004 | 602 | M |
| Dec. 6, 2004 | 602 | M |
| Dec. 19, 2004 | 589 | M |
| Jan. 2, 2005 | 575 | M |
| Jan. 2, 2005 | 575 | M |
| Jan. 2, 2005 | 575 | M |
| Jan. 2, 2005 | 575 | M |
| Jul. 4, 2005 | 392 | M |
| Jul. 4, 2005 | 392 | M |
| Sep. 3, 2005 | 331 | M |
| Sep. 3, 2005 | 331 | M |
| Sep. 24, 2005 | 310 | M |
| Sep. 24, 2005 | 310 | M |
| Sep. 24, 2005 | 310 | M |
| Oct. 23, 2005 | 281 | M |
| Oct. 23, 2005 | 281 | M |
| Oct. 23, 2005 | 281 | M |
| Oct. 23, 2005 | 281 | M |
| Nov. 15, 2005 | 258 | M |
| Nov. 15, 2005 | 258 | M |
| Aug. 17, 2004 | 713 | F |
| Aug. 17, 2004 | 713 | F |
| Aug. 17, 2004 | 713 | F |
| Aug. 18, 2004 | 712 | F |
| Aug. 18, 2004 | 712 | F |
| Aug. 21, 2004 | 709 | F |
| Aug. 21, 2004 | 709 | F |
| Aug. 21, 2004 | 709 | F |
| Aug. 21, 2004 | 709 | F |
| Sep. 10, 2004 | 689 | F |
| Sep. 11, 2004 | 688 | F |
| Sep. 11, 2004 | 688 | F |
| Oct. 2, 2004 | 667 | F |
| Oct. 2, 2004 | 667 | F |
| Nov. 5, 2004 | 633 | F |
| Nov. 5, 2004 | 633 | F |
| Nov. 5, 2004 | 633 | F |
| Nov. 5, 2004 | 633 | F |
| Nov. 5, 2004 | 633 | F |
| Feb. 2, 2005 | 544 | F |
| Feb. 2, 2005 | 544 | F |
| Feb. 2, 2005 | 544 | F |
| Feb. 2, 2005 | 544 | F |
| Feb. 2, 2005 | 544 | F |
| Jul. 4, 2005 | 392 | F |
| Jul. 4, 2005 | 392 | F |
| Jul. 4, 2005 | 392 | F |
| Aug. 20, 2005 | 345 | F |
| Aug. 20, 2005 | 345 | F |
| Aug. 20, 2005 | 345 | F |
| Aug. 20, 2005 | 345 | F |
| Aug. 20, 2005 | 345 | F |
| Sep. 3, 2005 | 331 | F |
| Sep. 3, 2005 | 331 | F |
| Sep. 3, 2005 | 331 | F |
| Sep. 3, 2005 | 331 | F |
| Sep. 24, 2005 | 310 | F |
| Sep. 24, 2005 | 310 | F |
| Sep. 24, 2005 | 310 | F |
| Oct. 23, 2005 | 281 | F |
| Oct. 23, 2005 | 281 | F |
| Oct. 23, 2005 | 281 | F |
| Oct. 23, 2005 | 281 | F |
| Nov. 15, 2005 | 258 | F |
| Nov. 15, 2005 | 258 | F |
| Nov. 15, 2005 | 258 | F |
| Nov. 15, 2005 | 258 | F |
| Nov. 15, 2005 | 258 | F |
| Nov. 15, 2005 | 258 | F |
| Nov. 15, 2005 | 258 | F |
| Nov. 15, 2005 | 258 | F |
| Nov. 15, 2005 | 258 | F |
| Nov. 15, 2005 | 258 | F |
| Dec. 2, 2005 | 241 | F |
| Dec. 2, 2005 | 241 | F |
| Dec. 6, 2005 | 237 | F |

Preliminary evaluations of food intake of the NPC1L1 −/− and wild-type mice were examined. There is no difference between the wild-type and NPC1L1 −/− mice. This indicates that lack of NPC1L1 (or inhibition) does not suppress apetite and therefore, it works via a mechanism proposed above.

A recent work (Pierpaoli et al., Proc. Natl. Acad. Sci. USA. 1994; 91: 787-91) reported the average life of C57B16 mice as 743±18.8 days (very close to the calculated average, above). In the Pierpaoli report, the goal was to increase mouse longevity by treating these mice with melatonin. Their results were deemed highly significant because they were able to extend the life of the C57B16 mouse to 871±30.5 days. It is notable that many of the mice of the present invention, i.e., the NPC1L1 −/−, have passed this point.

### Example 2

### Identification of NPC1L1 Inhibitors Using an Assay Based on Ricin Endocytosis

Following the observation that human liver has the highest expression of NPC1L1, we characterized the human liver derived cell line Hu7. Consistent with previous data of the present inventors, these cells express significant amounts of NPC1L1 as evidenced by mRNA and protein levels. These cells were chosen for subsequent studies.

Briefly, stable clones were generated that expressed higher levels of NPC1L1 by introducing the human NPC1L1 cDNA into these cells. About 30 clones were characterized, one of which (clone #3) had about a five-fold increase in NPC1L1 protein expression.

Next, a number of siRNAs were designed that targeted the NPC1L1 mRNA at various positions. Upon evaluation, two of these siRNAs were identified which targeted NPC1L1 very efficiently. Both of these siRNAs were inserted into a vector and were used to generate stable cell-lines. More than 50 of these cell lines were characterized, and four (4) were chosen for further characterization. Cell line designated si6 was the best. The si6 cell line exhibited greater than a 90% decrease in the NPC1L1 mRNA making this clone effectively null for NPC1L1 protein expression.

Lipid uptake experiments were carried out in the si6 cells and NPC1L1 positive clone #3 using various toxins to probe their transport. Briefly, fluorescent lipids (LacCer, cholesterol, and ceramide) were incubated with cells for 60 min at 4° C. and then chased at 37° C. for 30 min. All lipids exhibited altered uptake and localization, specifically to the Golgi apparatus, when compared between the NPC1L1 positive clone #3 and the negative si6 clone.

The endocytosis of a number of toxins such as ricin, diphtheria toxin and verotoxin was then tested based on previous observations. For all toxins, the si6 cells appear to target the toxins to the Golgi much more rapidly than either the wild-type cells or the clone #3 cells. Ricin exhibited the highest sensitivity. To confirm that these results are not due to something unique to clone si6 the experiments were repeated, using Ricin uptake, with other, independent siRNA clones. All clones with the exception of one (probably not a good siRNA clone) demonstrate the same results as above.

Next, a time course was used to determine the optimal time for detecting these differences. As early as 15 minutes following addition of the toxin, the difference in endocytosis in apparent. si6 cells show a dramatic Golgi staining with the toxin whereas the wild-type and #3 clone cells exhibit only a punctate type of staining.

The #3 clone and Ricin intoxication an assay was used to screen inhibitors by determining an increase in #3 clone's sensitivity to Ricin based on NPC1L1 inhibition. Several compounds were identified as potent NPC1L1 inhibitors as follows:

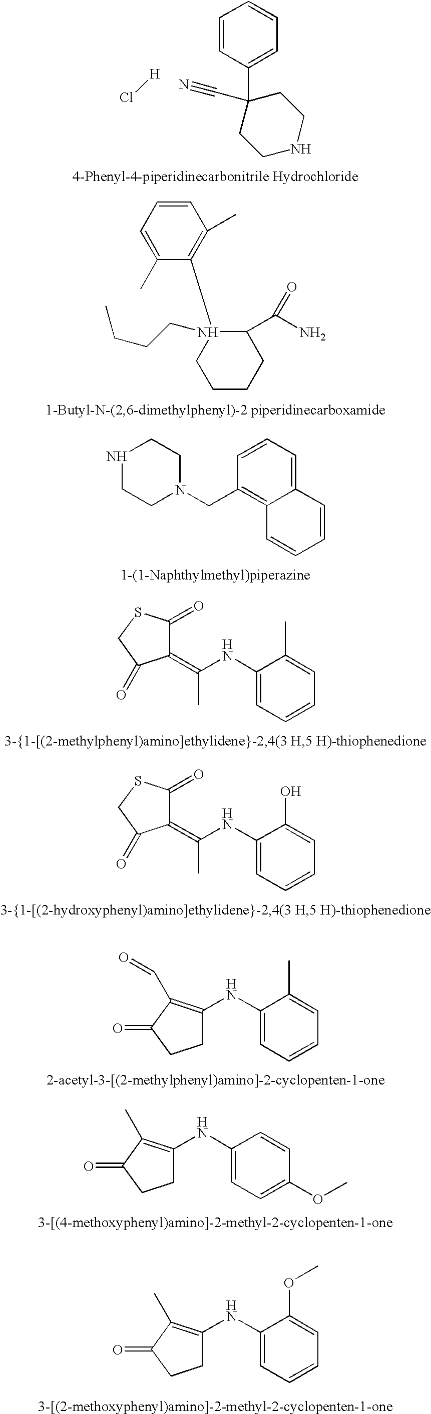

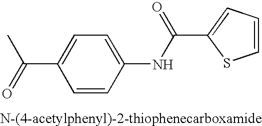

In addition, another inhibitor was identified (below) using a prokaryotic screening method.

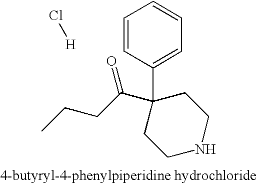

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, procedures, and publications cited throughout this application are incorporated herein by reference in their entireties.

What is claimed is:

1. A method for extending longevity in an individual comprising administering to the individual an effective amount of an NPC1L1 antagonist in a pharmaceutically acceptable carrier.

2. The method of claim 1, wherein the individual has not been diagnosed with a chronic disorder which adversely impacts longevity.

3. The method of claim 2, wherein the chronic disorder the individual has not been diagnosed with is hypercholesterolemia.

4. The method of claim 1, wherein the individual has been diagnosed with a chronic disorder which adversely impacts longevity.

5. The method of claim 4, wherein the chronic disorder is a cardiovascular disease.

6. The method of claim 5, wherein the cardiovascular disease is selected from the group consisting of hyperlipidemia, dyslipidemia, high cholesterol, and artherosclerosis.

7. The method of claim 1, wherein the NPC1L1 antagonist is ezetimibe (Zetia®).

8. The method of claim 7, wherein the ezetimibe (Zetia®) is administered at a dose of about 0.5 to 20 mg/day.

9. The method of claim 8, wherein the ezetimibe (Zetia®) is administered at a dose of about 5-15 mg/day.

10. The method of claim 9, wherein the ezetimibe (Zetia®) is administered at a dose of about 10 mg/day.

11. The method of claim 1, wherein the NPC1L1 antagonist is selected from the group consisting of an anti-NPC1L1 antibody, an NPC1L1 antisense nucleic acid, an NPC1L1 ribozyme, an NPC1L1 triple-helix, an NPC1L1 inhibitory RNA, or an NPC1L1 transcriptional inhibitor.

12. The method of claim 1, wherein the NPC1L1 antagonist is a 4-phenylpiperidine.

13. The method of claim 12, wherein the 4-phenylpiperidine is 4-phenyl-4-piperidinecarbonitrile hydrochloride, which has the following structure:

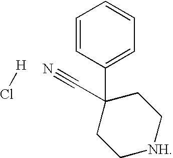

14. The method of claim 1, wherein the NPC1L1 antagonist is 1-butyl-N-(2,6-dimethylphenyl)-2-piperidinecarboxamide, which has the following structure:

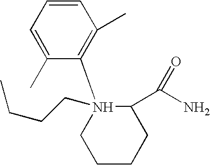

15. The method of claim 1, wherein the NPC1L1 antagonist is 1-(1-napthylmethyl)-piperazine, which has the following structure:

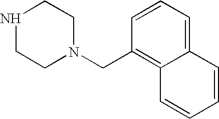

16. The method of claim 1, wherein the NPC1L1 inhibitor is 4-butyrl-4-phenylpiperidine hydrochloride, which has the following structure:

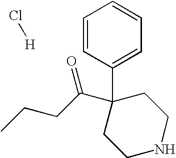

17. The method of claim 1, wherein the NPC1L1 inhibitor is 3-{1-[2-methylphenyl)amino]ethylidene}-2,4(3H,5H)-thiopenedione, which has the following structure:

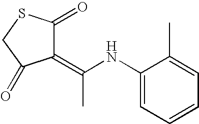

18. The method of claim 1, wherein the NPC1L1 inhibitor is 3-{1-[2-hydroxyphenyl)amino]ethylidene}-2,4-(3H,5H)-thiophenedione, which has the following structure:

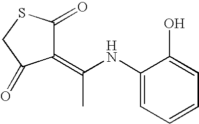

19. The method of claim 1, wherein the NPC1L1 inhibitor is 2-acetyl-3-[2-methylphenyl)amino]-2-cyclopenten-1-one, which has the following structure:

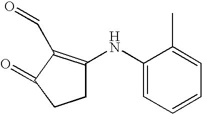

20. The method of claim 1, wherein the NPC1L1 inhibitor is 3-[4-methoxyphenyl)amino]-2-methyl-2-cyclopenten-1-one, which has the following structure:

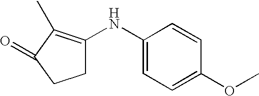

21. The method of claim 1, wherein the NPC1L1 inhibitor is 3-[2-methoxyphenyl)amino]-2-methyl-2-cyclopenten-1-one, which has the following structure.

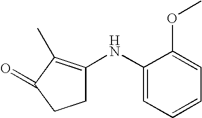

22. The method of claim 1, wherein the NPC1L1 inhibitor is N-(4-acetylphenyl)-2-thiophenecarboxamide, which has the following structure:

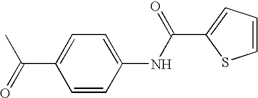

23. The method of claim 1, wherein the longevity is prolonged by at least 15% compared to the expected longevity of the individual, or as compared to an individual of similar expected longevity who has not been administered an NPC1L1 antagonist.

24. The method of claim 1, wherein the NPC1L1 antagonist is administered in combination with a second, longevity-prolonging agent.

25. The method of claim 24, wherein the second, longevity-prolonging agent is selected from the group consisting of deprenyl, melatonin, centrophenoxine, dehydroepiandrosterone (DHEA), synthetic human growth hormone, piracetam, vinpocetine-hydergine, procaine, centrophenoxine, phosophatidylserine, acetyl-L-carnatine, aspirin, and inhibitors of reactive oxygen intermediates.
